(19) United States
(12) Patent Application Publication
(10) Pub. No.: US 2022/0381766 A1
(43) Pub. Date: Dec. 1, 2022

(54) SYSTEM AND METHOD OF GAS SAMPLING FOR TRACE-LEVEL ANALYSIS OF CHEMICAL COMPOUNDS

(71) Applicant: Entech Instruments Inc., Simi Valley, CA (US)

(72) Inventor: Daniel B. CARDIN, Simi Valley, CA (US)

(21) Appl. No.: 17/804,557

(22) Filed: May 28, 2022

Related U.S. Application Data
(60) Provisional application No. 63/194,879, filed on May 28, 2021.

(51) Int. Cl. G01N 33/497; G01N 31/22; G01N 33/00

(52) U.S. Cl. CPC G01N 33/497; G01N 31/223; G01N 33/0047; G01N 2033/4975

(57) ABSTRACT

A hybrid gas sampling device can combine the functionality of both whole air and sorbent based samplers. The sampling device can be used for collecting light to very heavy organic compounds, for subsequent thermal desorption into a GC or GCMS for quantitative measurement. The sampling device isolates collected samples of gas phase matrices in a sample vessel, provided with sorbent elements from a removable sample extraction device. The sampling device is operated by drawing a vacuum on the chamber through the sample extraction device after sampling, and then completing the extraction of the heavier organic compounds using a static, diffusive extraction under vacuum to allow optimal deposition of the heavier compounds on the sorbent. The vacuum container is cooled to draw any excess water back into the container, thereby dehydrating attached sorbent element(s) in preparation for thermal desorption into a GC or GCMS, eliminating interferences in the MS analyzer.

### CROSS-REFERENCE TO RELATED APPLICATION

This application claims the benefit of U.S. Provisional Patent Application No. 63/194,879, filed on May 28, 2021, the entire disclosure of which is incorporated herein by reference in its entirety for all purposes.

### FIELD OF THE DISCLOSURE

The disclosure relates to chemical analysis and, more particularly, the collection of a gas sample and recovery of volatile organic compounds (VOCs) and semi-volatile organic compounds (SVOCs) for analysis by a GC or GCMS. Some embodiments of the disclosure relate to systems designed for being capable of gas sampling and compound recovery in applications containing high humidity levels, or even steam.

### BACKGROUND OF THE DISCLOSURE

Systems and processes for determining the levels of VOCs through SVOCs in gas phase matrices, such as indoor air, ambient air, workplace air, fenceline monitoring, refinery gas streams, and stack gas emissions, have been performed for decades. Such systems and processes have demonstrated numerous limitations in accurately measuring all compounds of interest at a wide range of concentrations.

In some examples, either permanent or portable real time analyzers have been used to perform these measurements, but virtually none of these “on-site” analyzers can handle high humidity samples while covering the large range of compound detection needed to measure all VOC through SVOC compounds present (which can include thousands, or even hundreds of thousands of compounds). In addition, prices for such analyzers can be prohibitive for many applications. In general, virtually all chemical analyzers demonstrate interferences when water reaches high PPM to percent levels in the sample. As an example, an excess of water in a sample for testing by GCMS can cause damage to the testing equipment (e.g., damage to the GC column) or suppressed response (e.g., due to lowered sensitivity of the MS).

Whole gas sampling devices can manage excess water by allowing it to condense out in the device—however, these approaches are accompanied by various shortcomings. First, many of the collected chemicals can react with condensed water if they are allowed to remain exposed to the water for prolonged durations of time awaiting analysis. Further, many SVOCs will stick to the walls of the container unless the containers are heated to higher temperatures to drive the low volatility compounds back into the gas phase. However, heating the container walls in this way can in turn also increase the amount of water vapor introduced back into the gas phase, again creating a problem with GCMS analysis. For many SVOCs, a relatively high temperature would be needed to transfer them substantially back into the gas phase, which would not only transfer far too much water into the analytical system but would cause many compounds to react in the presence of both a higher temperature and high water concentrations. Therefore, this problem with managing the temperature of the gas during sampling, the reduction of water before delivery to a GCMS, and the need to recover both VOCs and SVOCs have caused all prior sampling devices to fail at quantitative measurement under the sampling conditions ranging from ambient temperatures to 300 degrees Celsius, and moisture concentrations from 0 to 50 percent.

### SUMMARY OF THE DISCLOSURE

The disclosure relates to chemical analysis and, more particularly, the collection of a gas sample and recovery of volatile organic compounds (VOCs) and semi-volatile organic compounds (SVOCs) from the gas sample at low humidity levels through high humidity levels, as well as techniques for preparing the recovered compounds for analysis by a GC or GCMS (e.g., by enrichment, dehydration, concentration, and other techniques described below). In some examples, during the initial evacuation of a sample vessel after the sample has been collected, an “active sampling,” or Dynamic Headspace Sampling (DHS) method can be used. After the vacuum is formed and the vacuum source has been removed, any remaining transfer of the heavier SVOCs is done diffusively. Therefore, in some examples, a hybrid sampling system can operate in both active and passive (diffusive) modes, whereas other sampling systems can only operate in the active sampling mode, or DHS. In some examples a gas sampling system for recovering VOCs and SVOCs can describe an assembly of a sample vessel, vacuum sleeve, and sample extraction device. In some examples, a gas sampling system for recovering VOCs and SVOCs can describe an assembly without a sample extraction device. In such examples, a vacuum sleeve or other attachment can be used to couple a sample extraction device to the sample vessel (e.g., at a laboratory). In some examples, compound recovery from the gas phase matrix samples collected in the sample vessel can require a first sample extraction device that has sorbent element(s) optimized for VOCs through light SVOCs, and a second sample extraction device that has sorbent element(s) optimized for SVOCs.

In some examples, a breath sampling system is described, in which a breath sampler inlet has a high flow port that can be selectively used to evacuate a sample vessel, and to fill it with a final fraction of a patient's exhalation, to recover various chemicals and compounds both contained in the gas phase, and in the aerosols and/or droplets in a patient's breath.

### BRIEF DESCRIPTION OF THE DRAWINGS

FIGS. 1A and 1B illustrate example configurations for a gas sampling system for recovering VOCs and SVOCs according to some embodiments of the disclosure.

FIGS. 2A and 2B illustrate examples of sample extraction devices that can enrich a sample containing both VOCs and SVOCs prior to thermal desorption into a GC or GCMS according to some embodiments of the disclosure.

FIG. 3 illustrates an example of a sample extraction device placed in a lab isolation sleeve according to some embodiments of the disclosure.

FIGS. 4A and 4B illustrate examples of a hybrid sampling system including the sample extraction device for collecting gas samples and recovering VOCs and SVOCs according to some embodiments of the disclosure.

FIG. 5 illustrates an exemplary process for operating the hybrid sampling system to sample gas phase matrices using a single sample extraction device, and for maintaining conditions for compounds from the sampled gas to come into contact with sorbent element(s) of the sample extraction device according to some embodiments of the disclosure.

FIG. 6 illustrates an exemplary process for operating the hybrid sampling system to sample gas phase matrices using dual sample extraction devices according to some embodiments of the disclosure.

FIG. 7 illustrates an exemplary process for operating the hybrid sampling system to sample gas phase matrices using dual sample extraction devices and different compound recovery processes for different sample extraction devices according to some embodiments of the disclosure.

FIG. 8 illustrates an exemplary breath sampling system with a breath inlet in the “Flow Off” up position according to some embodiments of the disclosure.

FIG. 9 illustrates the exemplary breath sampler of FIG. 8 with the breath inlet in the “Flow Open” position to allow pre-evacuation of a sample vessel according to some embodiments of the disclosure.

FIG. 10 illustrates the exemplary breath sampling system with the breath inlet in the “Flow Divert” position coupled with a disposable mouthpiece attached in the up “Ready to Sample” position to allow for removal of an initial fraction of a patient's exhaled breath according to some embodiments of the disclosure.

FIG. 11 illustrates the exemplary breath sampling system with the breath inlet in the “Flow Open” position coupled with the disposable mouthpiece in the down “Collecting Breath” position to allow for sampling of a final fraction of a patient's exhaled breath according to some embodiments of the disclosure.

FIG. 12 illustrates the exemplary breath sampling system with the breath inlet and the disposable mouthpiece replaced with a sample extraction device according to some embodiments of the disclosure.

FIG. 13 illustrates an exemplary process for operating a breath sampling system to collect breath samples and recover compounds from the samples according to some embodiments of the disclosure.

### DETAILED DESCRIPTION

In the following description, reference is made to the accompanying drawings which form a part hereof, and in which it is shown by way of illustration specific examples that can be practiced. It is to be understood that other examples can be used, and structural changes can be made without departing from the scope of the examples of the disclosure.

The disclosure relates to chemical analysis and, more particularly, the collection of a gas sample that may or may not contain condensing droplets of moisture in the gas stream, and subsequent recovery of volatile organic compounds (VOCs) and semi-volatile organic compounds (SVOCs) from the gas sample across a wide range of moisture or humidity levels, and at any initial gas phase sample temperature from 0 to 300 degrees Celsius, as well as techniques for preparing the recovered compounds for analysis by a GC or GCMS (e.g., by enrichment, dehydration, concentration, and other techniques described below. Embodiments of the present disclosure allow for sampling and recovery of VOCs and SVOCs at a field or site of interest (e.g., refinery gas streams, process gas streams, stack gas emissions, work place air, fenceline monitoring, ambient air, indoor air, and other gas phase matrices). Embodiments of a sampling process according to the present disclosure can include active collection of a gas phase matrix samples at a site of interest, using a pre-evacuated sample vessel (e.g., a vessel in which a vacuum has been drawn), in some examples. The pre-evacuated sample vessel can have a volume, corresponding to the required detection limits for the particular application of any given example or embodiment. At the time and/or location of sample collection, a sample extraction device can be optionally integrated into a sampler to allow for at least a portion of samples collected in a vessel to be recovered onto the sample extraction device (e.g., while additional samples continue to be collected, or during transfer to the analysis laboratory), in some examples. In some examples, a sample vessel can be used to collect samples without any sample extraction device(s) present at the collection site/field, and is later coupled to the sample extraction device(s) at a later time when the sample vessel is returned to a lab environment for analysis of its contents.

The current approach described by embodiments of the disclosure allows VOCs, SVOCs, organic compounds, and any number of compounds to be collected onsite (often referred to as “in the field”), without the need for a co-present analyzer (thereby reducing cost and complexity, and improving the range and bandwidth of compounds at the site that can be analyzed off-site). Specifically, embodiments of the disclosure allow all VOCs and SVOCs of interest to be recovered, while minimizing water content of the final extracted sample prior to GCMS analysis. Virtually all gas phase matrices can be sampled by the approach described by embodiments of the disclosure, although the approach described below is particularly well-suited towards collecting light VOCs thru heavy SVOCs in either elevated temperature gas streams, or in high moisture content streams (e.g., streams at 50% or more water vapor). The sampler described by embodiments of the disclosure is uniquely capable in collecting stack gas for analysis of a wider range of compounds than alternative approaches.

The sampler can also handle small aqueous droplets and aerosols, making it ideal for collecting breath samples for GCMS analysis. Aerosols in breath samples have created problems with other alternative sampling techniques, both because the chemicals of interest in the aerosols have difficulty reaching the analyzer and/or can cause undesirable contamination of the sampling device(s) meant only for volatile chemical adsorption and desorption. Aerosols in breath samples can contain proteins, carbohydrates, lipids, bacteria, and other non-volatiles that when thermally desorbed (e.g., at temperatures in the range of 200-300 degrees Celsius) would break apart into artifact compounds that were never present in the original sample. These artifact compounds that can be generated by decomposition caused by the thermal desorption will nonetheless show up in the GCMS analysis, thereby complicating the question as to the actual contents and/or identities of the compounds present in the actual, original sample (e.g., the sample collected by the sample vessel in the field).

In some embodiments, environmental gas phase matrix samples can be collected into sample vessel over a time period (e.g., a sampling period), based on an adjustable flow restrictor or controller coupled to an inlet attached to the sample vessel, as described in greater detail below. In some embodiments, high flow breath samples can be collected on the order of just a few seconds (e.g., 8-10 seconds, 10-12 seconds, etc.) by using an alternate design with a sample inlet that can be pushed into, and pulled out of the sample vessel, as described in greater detail below.

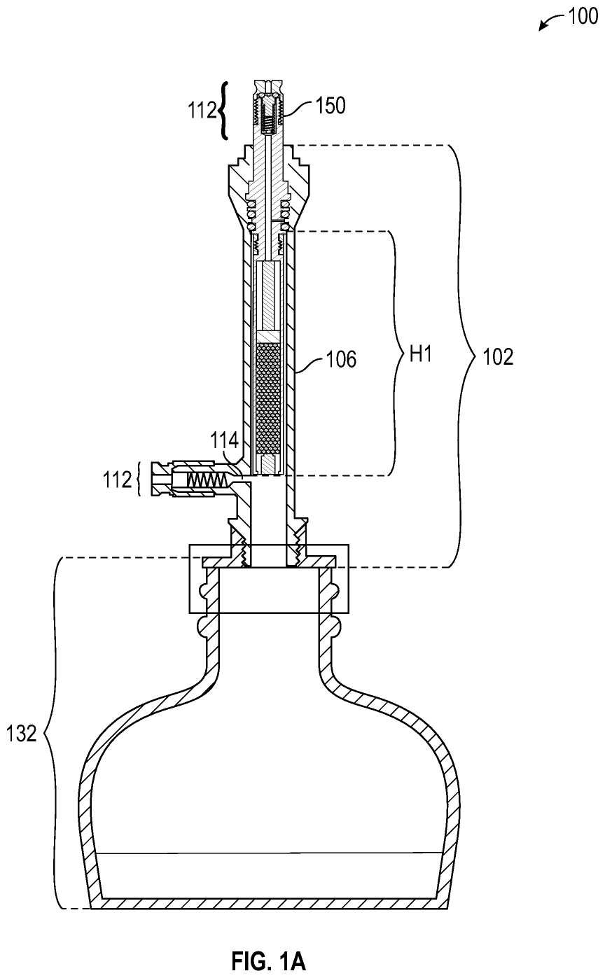

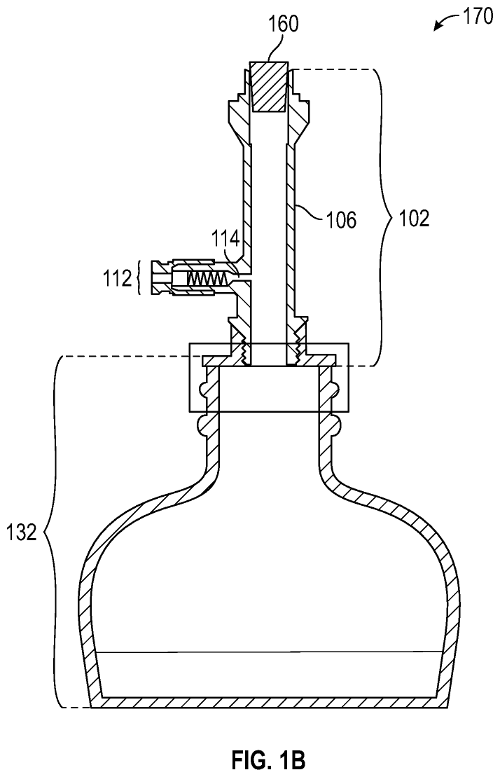

FIGS. 1A and 1B illustrate example configurations for a gas sampling system for recovering VOCs and SVOCs according to some embodiments of the disclosure. FIG. 1A particularly illustrates a cross-sectional side view of a hybrid sampling system 100 including a sample extraction device 150 for sampling gas phase matrices and collecting VOCs and SVOCs from the samples according to some embodiments of the disclosure. System 100 illustrates an embodiment in which a sample collection device (e.g., sample vessel 132) is co-present with, and coupled to a sample extraction device (e.g., device 150). The configuration of system 100 (FIG. 1A) can be used to collect a gas phase sample having a range of humidity levels (e.g., from low levels of humidity to high levels of humidity, including gas phase matrices that contain steam), and to substantially recover VOCs and SVOCs contained in the samples of the gas phase matrix, during a sampling interval. In some embodiments, vacuum sleeve 102 has a side port 112 corresponding to a valve that allows gases to travel in and out of sample vessel 132, through inlet 114. A vacuum can be created within sample vessel 132 using side port 112 to remove preexisting gases (e.g., clean nitrogen original in the vessel) to provide the vacuum needed to draw in the gas stream during sample collection. In some embodiments, bulk gases corresponding to samples contained within the inner chamber of sample vessel 132 can be removed through side valve 114 of sample extraction device 150, causing those gases and some compounds contained in the samples to come into contact with one or more sorbent elements (illustrated without reference numerals in FIG. 1A) positioned near a lower opening of sample extraction device 150.

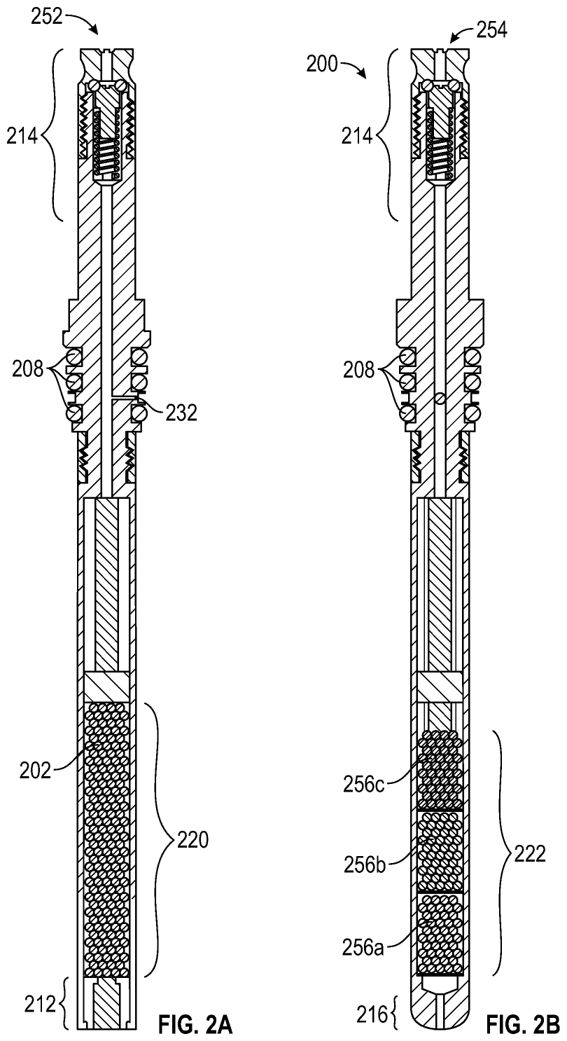

Sample extraction device 150, described in greater detail below in connection with FIGS. 2A-2C, generally has a lower opening positioned toward sample vessel 132, such that contents within sample vessel 132 (e.g., VOCs and SVOCs) can enter the lower opening of sample extraction device 150 according to the sample recovery processes described in greater detail below. For simplicity of illustration, sample extraction device 150 is illustrated as being inserted into vacuum sleeve 102, which is connectively coupled to sample vessel 132. Sample vessel 132 can represent a vacuum sampling canister made of either glass or ceramic deactivated stainless, that can be used to quickly capture a sample. Conventional or alternative approaches to using a vacuum sampling canister to capture and analyze SVOCs have been found to be ineffective, because the SVOCs can be lost onto the internal surface of the vacuum sampling canister.

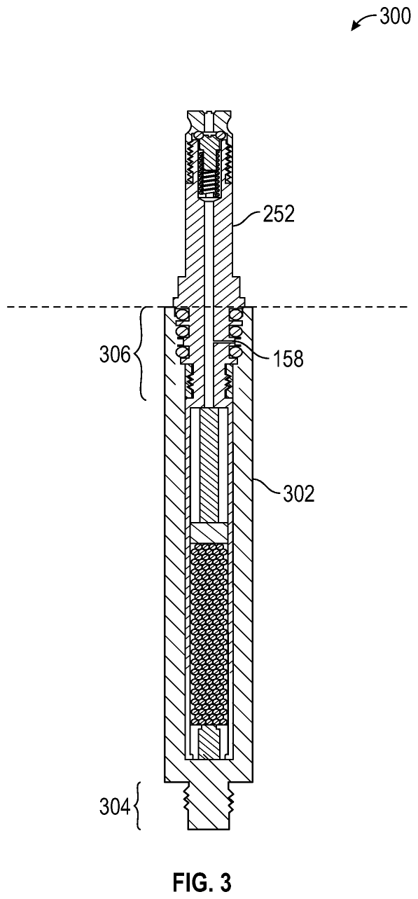

Vacuum sleeve 102, in some examples, can have a shaft portion 106, which is configured to hold or position a portion of sample extraction device 150 (e.g., the portion of the device 150 corresponding to height H1) at a specific position relative to an opening of sample vessel 132 and/or inlet 114 of side port 112. As illustrated by system 100 (and FIGS. 3A and 3B), sample extraction device 150 extends to a location above an opening of inlet 114 connecting the inlet to the shaft portion 106.

FIG. 1B illustrates a cross-sectional side view of a hybrid sampling system 170 that does not include any sample extraction device, and is configured to sample gas phase matrices. System 170 illustrates an embodiment in which a cap attachment 160 is coupled to an upper opening of vacuum sleeve 102, and can correspond to an embodiment where system 170 is used to collect samples of gas phase matrices into sample vessel 132. Cap attachment 160 can, in some embodiments, be replaced with sample extraction device 150 as shown in FIG. 1A, once system 170 has collected its samples of a gas phase matrix (e.g., either on-site, or in a lab environment where the sample vessel is taken after sampling).

This approach can be used when performing classical dynamic headspace sampling to pull a known volume from sample vessel 132 through sample extraction device 150 (which is introduced in the lab). This approach can also be applied when extraction device 150 is added in the lab, followed by creation of a vacuum to extract both VOCs and SVOCs from both the gas phase and off the interior walls of vessel 132. System 170 of FIG. 1B may be used to reduce the cost of the sampling device sent out to the field, or in some cases to reduce the complexity of the device by non-lab personnel.

The configuration of system 170 illustrated by FIG. 1B can correspond to a system that is configured in a lab to solely sample gas phase matrices having a range of humidity levels (e.g., from low levels of humidity to high levels of humidity, including gas phase matrices that contain steam). System 170 can then be used on-site or in the field, to collect samples of gas phase matrices. System 170 can then be used, at a later time, to at least partially recover VOCs and SVOCs contained in the samples of the gas phase matrix, during a sampling interval (e.g., when cap attachment 160 is replaced by a sample extraction device 150, as shown by FIG. 1A). Whether or not extraction device 150 is inserted before, or after the gas phase matrix sampling process, the entire system (e.g., system 100 or 170) must be analytically cleaned and void of all background VOCs and SVOCs so as not to contaminate or positively bias the results.

In such configurations of system 100, the gas phase matrix sampling by sample vessel 132, and VOC/SVOC recovery by the sample extraction device 150 can occur during non-overlapping intervals (e.g., compounds can be recovered by device 150, at a later time from when samples are collected by vessel 132). In some embodiments, vacuum sleeve 104 has side port 112 corresponding to a valve that allows gases to travel in and out of sample vessel 132, through inlet 114. In some embodiments, an inner chamber of sample vessel 132 can be evacuated of any gases, through side port 112, to form a vacuum prior to collection of samples from a gas phase matrix.

FIGS. 2A and 2B illustrate examples of sample extraction devices 252 and 254 that can enrich a sample containing both VOCs and SVOCs prior to thermal desorption into a GC or GCMS according to some embodiments of the disclosure. Referring to FIG. 1A, sample extraction device 150 can correspond to any of devices 252 and 254, in various embodiments. Starting with FIG. 2A, sample extraction device 252, a device that can sample actively and/or diffusively, can have a diameter between 1/32 in. and ⅜ in. (e.g., the external or internal diameter of the sample extraction device 252). In some examples, other dimensions are possible. Sample extraction device 252 can comprise a tube-like structure, for example, that includes various channels and/or cavities as will be described below. In some examples, sample extraction device 252 can be fabricated from stainless steel or another suitable material (e.g., a material that is substantially inert).

Sample extraction device 252 can include lower cavity 220. In some examples, the lower cavity 220 can contain one or more sorbent element(s) 202, which can include, for example, an adsorbent material. As will be described below, in some examples, sorbent element(s) 202 can be selected to collect a sample for analysis. In some examples, the sorbent element(s) 202 can be located towards an extraction end 212 of the sample extraction device 252. That is to say, sorbent element(s) 202 can be closer to the extraction end 212 of the sample extraction device 252 than it is to a valve end 214 of the diffusive extraction device. During sample extraction, extraction end 212 of the sample extraction device 252 can be open to the environment of the sample vessel (e.g., sample vessel 132 of FIGS. 1A and 1B) such that the sample being extracted or collected can enter lower cavity 220, and can adsorb or absorb to sorbent element(s) 202, as will be described in more detail below. In some embodiments, sample extraction device 252 can be used to perform diffusive sampling of a sample vessel, in which compounds come into contact with sorbent element(s) 202 based on diffusion of the compounds. In some embodiments, the diffusive sampling occurs in the absence of a vacuum being actively applied to any part of the sampling system using the sample extraction device. In some embodiments, the diffusive sampling process occurs in a closed system. In some embodiments, the closed system is under vacuum (e.g., is evacuated).

At the valve end 214 of the diffusive extraction device 252 (e.g., opposite extraction end 212 of the diffusive extraction device 252), the diffusive extraction device 252 can include a sealing plunger, a spring, and an internal seal, for example. In some examples, the sealing plunger and internal seal can selectively restrict fluid (e.g., gas, liquid, etc.) flow through an internal channel between the sealing plunger or internal seal, and lower cavity 220 where sorbent element(s) 202 are located. For example, when the sealing plunger is pressed up against the seal, fluid flow through sample extraction device 252 can be restricted, and when the sealing plunger is moved away or otherwise separated from the seal, fluid flow through sample extraction device 252 may be unrestricted. In some examples, sealing plunger can be tensioned via a spring against the seal such that in a default configuration, the sealing plunger can be pressed up against the seal, thereby restricting fluid flow through sample extraction device 252. Fluid flow (e.g., air being drawn into a vacuum source) through sample extraction device 252 can be allowed by causing the sealing plunger to move away from the seal (e.g., via mechanical means such as a pin from above, or other means). For example, a vacuum source can be coupled to the sample extraction device 252 at the valve end 214 to open the sealing plunger and draw a vacuum through the sealing plunger, internal channel, and lower cavity 220. Additionally, in some examples, the sealing plunger can remain open (e.g., during continuous vacuum evacuation) to evaporate unwanted matrix, such as water or alcohol, from the sample through sorbent element(s) 202. In some embodiments, sample extraction device 252 can be used to perform active sampling of a sample vessel, sometimes referred to as Dynamic Headspace Sampling (DHS), in which compounds come into contact with sorbent element(s) 202 based on a vacuum source drawing a volume of gas through device 252. In some embodiments, the entire sampling system can be chilled before performing DHS, to trap lighter compounds, and to overall reduce the amount of water in the gas phase.

As an example, during a sample extraction process in which a sample can be collected in sample extraction device 252, a vacuum can be drawn through the sealing plunger, internal channel, and lower cavity 220 to facilitate sample collection by sorbent 202 in lower cavity 220. After the sample has been collected by the dynamic/diffusive extraction device 252, the sealing plunger can be remain closed (e.g., as it can be during sample collection) and can isolate the sample from the environment, allowing the sample to be stored in the sample extraction device 252 between extraction and analysis. For example, a spring can cause the sealing plunger to remain closed in the absence of a mechanical force to open the sealing plunger.

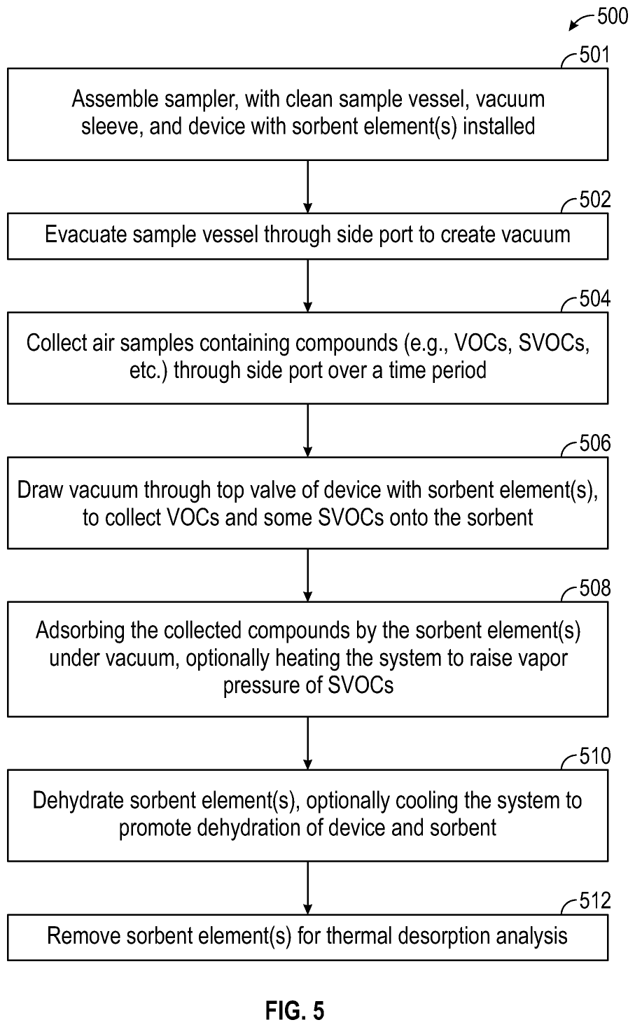

The sample extraction device 252 can further include one or more external seals 208, for example. The external seals 208 can be made of an elastomeric material and can be fluoroelastomer seals or perfluoroelastomer seals. The external seals 208 can be located externally on sample extraction device 252 between ends 212 and 214. The external seals 208 can include one or more gaskets or O-rings fitted around the outside of the sample extraction device 252, for example. In some examples, the external seals 208 can be used to form a seal between sample extraction device 252 and a sample vessel/vial into which sample extraction device 252 can be inserted during a sample extraction process, and/or to form a seal between sample extraction device 252 and a desorption device into which sample extraction device 252 can be inserted during a sample desorption process (e.g., while directing a desorb gas flow through the internal sorbent 106 during thermal desorption to an analyzer such as a GC or GCMS), such as part of the sample recovery processes described below with reference to FIGS. 5-7.

FIG. 2B illustrates a sample extraction device 254 similar to sample extraction device 252. Sample extraction device 254 can sometimes be referred to as a Dynamic Headspace Sampling device that uses active sampling to draw a known volume of sample through the sorbent bed(s) during sample extraction. The small inlet associated with device 254 may not allow effective diffusive sampling as does 252, which can be used as an advantage when a delay in sampling is needed when device 254 is exposed to a gas containing VOCs and/or SVOCs.

A notable difference between sample extraction device 252 and sample extraction device 254 can be the length of the sorbent bed (e.g., the regions within cavity 220 and cavity 222 containing sorbent element(s)). In some examples, the longer adsorbent bed of sample extraction device 254 can accommodate a plurality of sorbent element(s) 256a-c, accessible via an opening 214. In some embodiments, where sample extraction device 254 is used in conjunction with active sampling processes, channeling can be a bigger problem, requiring a longer adsorbent bed being used in sample extraction device 254, relative to sample extraction device 252, to prevent breakthrough losses of some compounds. In some embodiments, an opening 214 of an extraction end of sample extraction device 254 shown in FIG. 2B can have a diameter that is substantially smaller than an opening of extraction end 212 shown in FIG. 2A (e.g., a diameter less than ⅜ in.). Smaller opening 214 of an extraction end of sample extraction device 254 of FIG. 2C can render sample extraction device 254 particularly well-suited to active sampling, during which a mechanical force to open a sealing plunger within valve end 214 is applied, and gases are pulled through sample extraction device 254 during a sample extraction process in which a sample can be collected, by a vacuum be drawn through a sealing plunger located at valve end 214 (e.g., when forced open by the mechanical force).

In some embodiments, sorbent element(s) 256a-c can be arranged in order of increasing chemical affinity to one or more compounds of interest in a sample. For example, sorbent 256a can have a relatively low chemical affinity, sorbent 256b can have a higher chemical affinity than sorbent 256a, and sorbent 256c can have the highest chemical affinity of the sorbent element(s). Sample extraction devices 252 and/or 254 can collect the sample using an active flow of gas, a static diffusive flow, and/or by sealing the sample extraction devices 252 and/or 254 inside of a vessel/vial under vacuum. In some embodiments, pulling a vacuum on the sample while the sample extraction device 252 and/or 254 is sealed in the sample vessel/vial containing the sample can improve the rate of extraction, especially for the extraction of the higher boiling point compounds. In some embodiments, sample extraction device 252 is connectively coupled to a sample vessel/vial throughout a gas matrix sampling interval, during which air/gas samples are collected directly into a sample collection vial, and indirectly collected/concentrated onto the sorbent(s) of the sample extraction device by a static diffusive flow caused by the random movement of molecules within a sample vial (or other suitable vessel for collecting a sample of a gas matrix). In some embodiments, sample extraction device 254 can be inserted into a vacuum sleeve just after sample collection. In some embodiments, sample extraction devices 252 and 254 are kept separate from a sample-collection vessel, sometimes referred to as simply a “sample vessel,” until after a gas phase matrix has been sampled by the sample vessel during the gas matrix, and prior to testing the compounds (e.g., at a testing site that is different than the sampling site or gas collection site).

FIG. 3 illustrates an example of a sample extraction device 252 placed in a lab isolation sleeve 302 according to some embodiments of the disclosure. For simplicity of illustration, sample extraction device 252 is illustrated as being inserted into isolation sleeve 302. However, it should be understood that isolation sleeve 302 can be used to hold sample extraction device 254 of FIG. 2B or any other suitable configuration of a sample extraction device, according to similar principles illustrated in connection with sample extraction device 252 of FIG. 2A being held by isolation sleeve 302 in FIG. 3. As described above in connection with FIG. 2A, external seals 208 are disposed around the outside of the body of the sample extraction device 252. As illustrated by FIG. 3, port 232 can be between external seals 208, which enables selectively sealing the port 232, is sealed by the insertion of sample extraction device 254 into isolation sleeve 302. Sleeve region 306 can represent a portion of isolation sleeve 302 with inner surface dimensions sized such that seals 208 (not labeled in FIG. 2D) are in contact with the inner surface of isolation sleeve 302. Threaded region 304 can represent a lower portion of isolation sleeve 302 that allows for the isolation sleeve to be screwed in and fastened to any compatible threads (e.g., on lab equipment such as a tray or extraction system for sample collection devices 252 and/or 254).

Isolation sleeve 302 not only protects the samples collected with the sorbent(s) of sample extraction device 252 after device 252 has been used to collect compounds from sampled gases in a sample vessel (e.g., a “post-sampling” state), but sleeve 262 also keeps the device 252 clean after thermal desorption or thermal conditioning (e.g., a “clean,” or “post-desorption” state). After desorption or cleaning, the device 252 can be returned to sleeve 262 with minimal exposure to air, because exposure to air can cause appreciable collect organic compounds from the air to collect onto the sorbent element(s) of device 252. Sleeve 262 therefore keeps devices 252 and/or 254 clean after desorption or cleaning (e.g., in the “clean” or “post-desorption” state), and maintains the integrity of compound samples collected on the sorbent element(s) of the devices (e.g., in the “post-sampling” state).

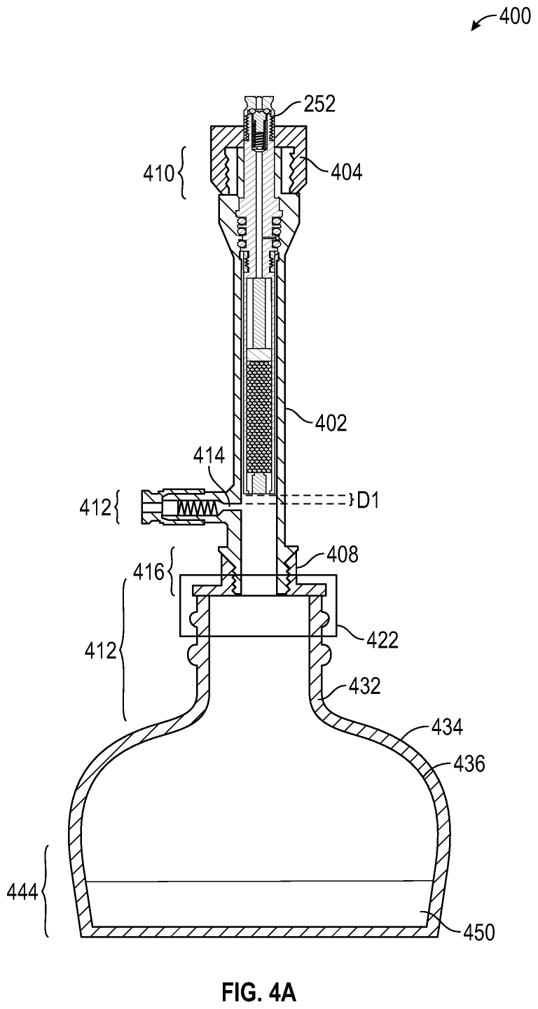

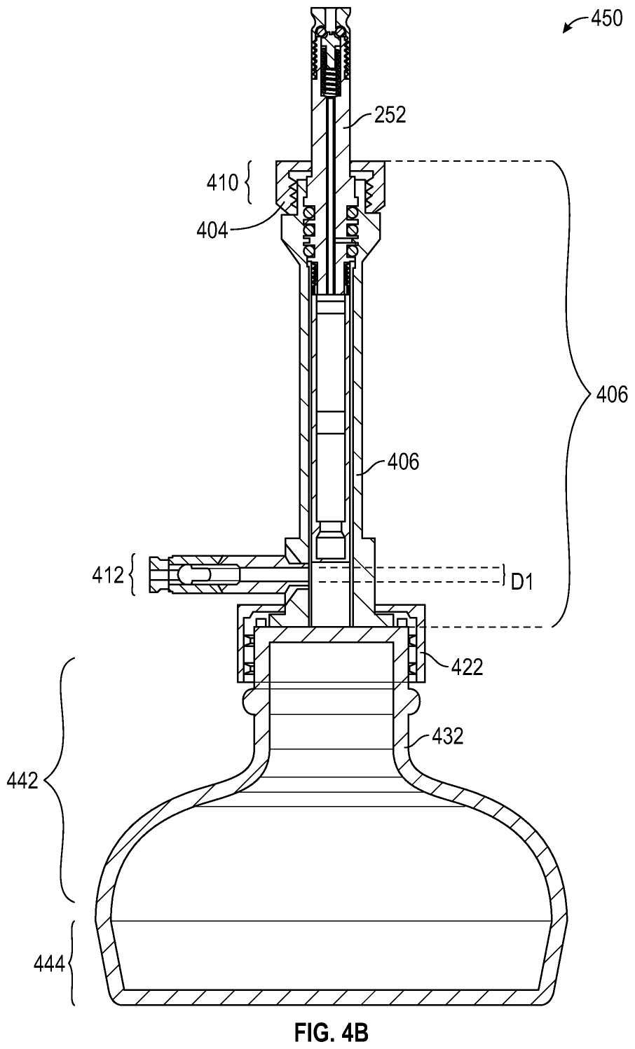

FIG. 4A illustrates a cross-sectional side view of a hybrid sampling system 400 including sample extraction device 252 for sampling gas phase matrices and collecting VOCs and SVOCs from the samples according to some embodiments of the disclosure. Similar to FIG. 1A, the “hybrid sampler” of FIG. 4A can be used to implement a sampling technique that is well-suited for both VOCs and SVOCs, unlike conventional or alternative approaches using a vacuum sampling canister that is susceptible to trapping SVOCs (e.g., preventing the SVOCs from being extracted and/or recovered by a sample extraction device). With a known volume of the sample vessel 432, the accuracy of the collected volume of gas phase matrix samples can be exact and/or repeatable every time. Volumetric sample injection of a small gas volume into a GC system can sometimes be referred to as “loop injection,” due to GC system tubing often being wound into a coil of round loops, and is one of the most reproducible sample injection techniques that typically can achieve a +−1% Relative Standard Deviation on multiple injections. In other words, volumetric sample determination has been proven to be very quantitative in nature, and lends itself ideally as a way to accurately determine the volume sampled that will ultimately transfer to the analyzer, minus any non-volatiles or excess water.

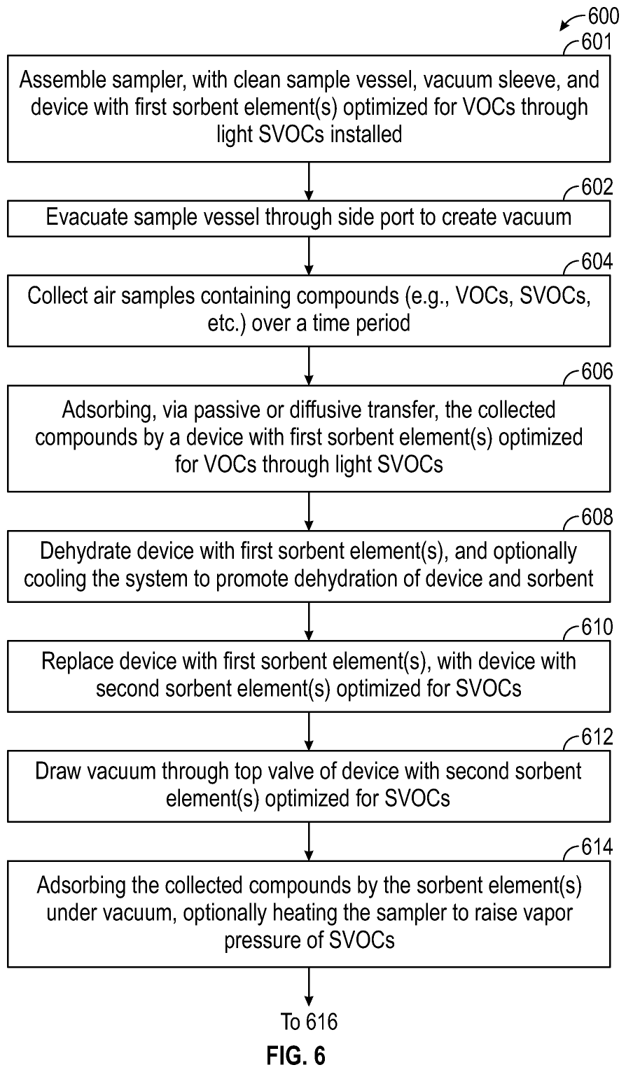

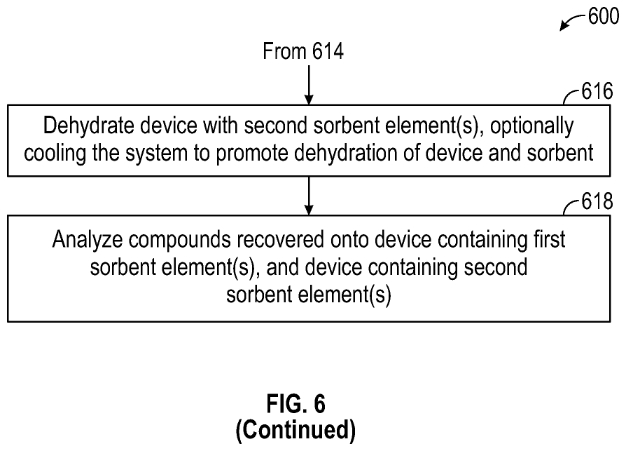

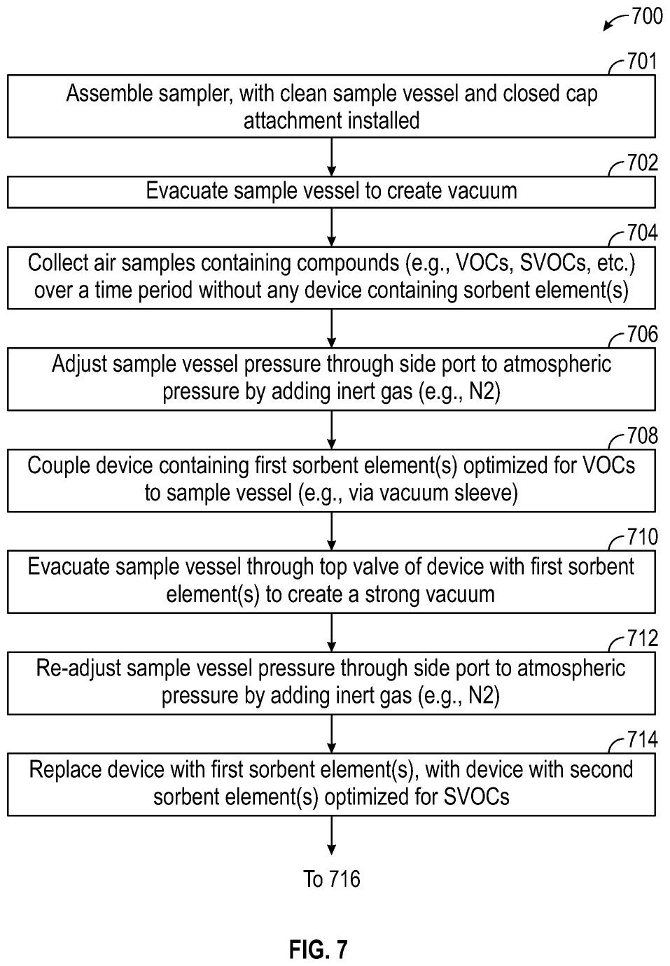

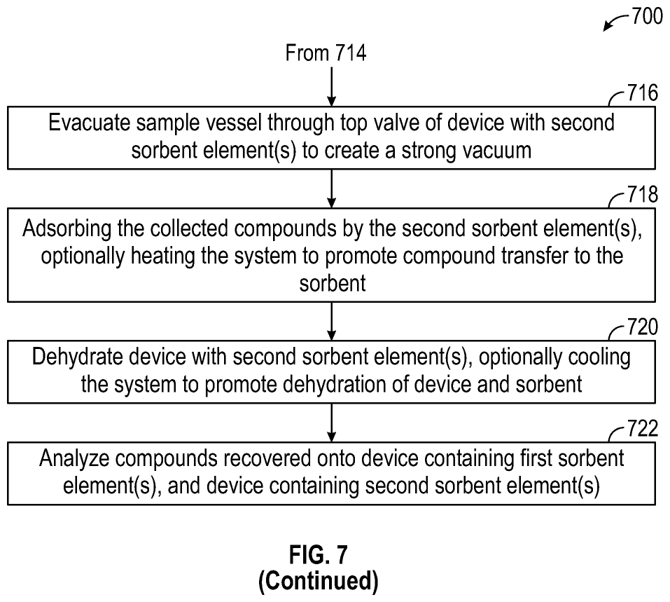

For simplicity of illustration, sample extraction device 252 is illustrated as being inserted into vacuum sleeve 402, and held in place within the vacuum sleeve 402 by retention cap 404 (which is attached to, and separable from vacuum sleeve 402 by compatible threading on contacting surfaces of the two components). In additional embodiments, hybrid sampling system 400 can be operated to sample gas phase matrices from the surrounding or ambient environment, or any other gas source provided at side port 412 by an adapter (e.g., an adapter to couple side port 412 to a refinery stack, an adapter to couple side port 412 to a ventilator to collect breath samples without a patient's direct participation, etc.), whether or not sample extraction device 252 and/or 252 are placed in vacuum sleeve 402. In other words, hybrid sampling system 400 can be operated to collect samples of complex gas phase matrices, without requiring sample extraction device 252 to be inserted in vacuum sleeve 302, or even present at the sampling site/location. Additionally or alternatively in some situations, hybrid sampling system 400 can be operated to recover compounds using more than one sample extraction device 252, as described in further detail below in connection with FIGS. 6 and 7. In some embodiments, operating hybrid sampling system 400 using more than one sample extraction device can allow more of the sample within the sample vessel to be collected, without reaching a breakthrough volume of compounds on the sorbent element(s) of any one sample extraction device. Breakthrough volume can refer to a volume that will cause particular compounds to be passed all the way through a sorbent bed (e.g., as opposed to being adsorbed and retained onto the sorbent bed). Breakthrough volume is a function of multiple factors, such as the sorbent strength, the particular compound's affinity to that adsorbent, and the temperature of the sorbent.

FIG. 4A illustrates sample extraction device 252 vertically offset from a side port 412 of vacuum sleeve 402 according to some embodiments of the disclosure. In particular, cavity 220 of sample extraction device 252 (e.g., an open end of the sample extraction device containing one or more sorbent elements) can be positioned within a shaft portion of vacuum sleeve 402 such that it is located above an opening of sleeve inlet 414, when seated within the vacuum sleeve. In some embodiments, sample extraction device 252 can be positioned such that its cavity containing the sorbent elements is vertically offset (or simply, above) sleeve inlet 414 by a distance D1. As an example, distance D1 can be 1 mm, 1 cm, or any suitable vertical offset for ensuring that a path exists for gases traveling through sleeve inlet 414 that does not come into contact with sample extraction device 252, before reaching the inner cavity of sample vessel 432 defined by inner sidewall 436. In additional embodiments, hybrid sampling system 400 can be operated to sample gas phase matrices from the surrounding or ambient environment, or any other gas source provided at side port 412 by an adapter (e.g., an adapter to couple side port 412 to a refinery stack, a mouthpiece adaptor for receiving a patient's exhaled breath, an exhaled breath line of a ventilator, etc.), whether or not sample extraction device 252 and/or 254 is placed in vacuum sleeve 402. In other words, hybrid sampling system 400 can be operated to collect samples of complex gas phase matrices, without requiring sample extraction device 252 to be inserted in vacuum sleeve 402, or even present at the sampling site/location.

In the embodiment illustrated by FIG. 4A, sample extraction device 252 is vertically offset from side port 412 and inlet 414 by a distance D1, such that gases traveling in/out of the sample vessel do not come into contact with exterior sidewalls of the sample extraction device 252. The embodiment illustrated by FIG. 4A is particularly well-suited to applications where hybrid sampling system 400 is used to sample gases from a gas phase matrix with any moisture content level (e.g., from low levels to high levels). In the arrangement of FIG. 4A, sample extraction device 150 does not require wiping down or sanitation before desorption and/or analysis (e.g., because any moisture/particles from a complex gas phase matrix with high water vapor content do not come into contact with exterior surfaces of sample extraction device 252, when traveling through inlet 414). In embodiments where the gas phase matrix is a very high water-content gas stream, an inlet 414 (sometimes referred to as “a minimal path length inlet”) with a small opening may be susceptible to clogging by condensing water vapor, were it not to be heated, for example. In some embodiments, the low thermal mass of inlet 414, which is substantially disconnected thermally from the rest of sampling system 400, would be easy to heat. to at least get the sample into the shaft portion of the vacuum sleeve 402, from where the sample can drip into the vessel.

Vacuum sleeve 402 can pneumatically couple or connect sample extraction device 252 to sample vessel 432 in some embodiments. Both vacuum sleeve 402 and sample vessel 432 can be formed from inert, non-absorptive, and non-adsorptive material in some embodiments (e.g., glass, stainless steel, ceramic coated stainless steel, etc.), ensuring that collected or sampled compounds will not interact or react with their respective surfaces. In some embodiments, certain compounds may potentially adsorb to surfaces of vacuum sleeve 402 and/or sample vessel 432 at lower temperatures in the range of 10 to 40 degrees Celsius. However, in such embodiments, such compounds are reintroduced into the gas phase during vacuum processing in a lab setting (e.g., at elevated temperatures). In some embodiments, both vacuum sleeve 402 and sample vessel 432 are formed from stainless steel. However, as mentioned above, any inert, non-absorptive, and non-adsorptive material that can form an inner cavity capable of maintaining a vacuum can be used to form vacuum sleeve 402 and/or sample vessel 432 in some embodiments. The cross-sectional side view of FIG. 4A illustrates sample extraction device 252 being held in place within vacuum sleeve 402 by an inner surface with dimensions sized such that seals 208 (not labeled in FIG. 4A) are in contact with the inner surface of vacuum sleeve 402. In addition to having an inner surface with dimensions to accommodate seals 208, vacuum sleeve 402 can have a threaded region 410 containing threads compatible with a retention cap 404.

Retention cap 404 can further secure sample extraction device 252 within vacuum sleeve 402, and further seal parts of the body of the sample extraction device to the ambient environment/air around the hybrid sampling system 400. Retention cap 404 prevents the sample extraction devices 252 from coming out of vacuum sleeve 402 until the cap is removed, in some embodiments. In embodiments that involve operations in the lab, cap 404 is not used, because it interferes with the operation of automated rail sampling devices (e.g., an autosampler) that automatically removes sample extraction devices 252 and 254 from their respective isolation sleeves 302, illustrated by FIG. 3. Although retention cap 404 is illustrated by FIG. 4A as having a cavity or opening in/through which sample extraction device 252 is seated or placed, some embodiments can include alternative geometries for the cap surface of retention cap 404. As an example, in some embodiments, retention cap 404 can have a solid surface free from any openings, such that when retention cap 404 is screwed onto threaded region 410, none of the environmental/ambient air surrounding the top portion of vacuum sleeve 402 can enter a shaft portion of the vacuum sleeve 402. In this way, retention cap 404 can seal a top portion of vacuum sleeve 402 to the environment, in embodiments where hybrid sampling system 400 is used without a sample extraction device 252 inserted.

In embodiments where retention cap 404 is a solid sampling cap (e.g., without a sample extraction device 252 as illustrated, similar to cap 160 of FIG. 1B), an additional approach is available to analyze VOCs through SVOCs. VOCs can generally remain in the gas phase, whereas SVOCs can primarily go onto the surface of the vacuum sample vessel 432. In the lab, the pressure inside of sample vessel 432 can first be equilibrated to atmospheric pressure using UHP Nitrogen injected into the vessel via side port 412, so a sample extraction device 252 that is optimized for VOC recovery using Dynamic Headspace Sampling techniques can be inserted, once cap 404 has been removed. VOC sampling onto a TD tube can then be used to collect a fraction of the contents of sample vessel 432 for VOC analysis, so that a known amount of the sample is delivered through sample extraction device 252. Then, sample vessel 432 can be brought back up to atmospheric pressure (e.g., using UHP Nitrogen injection) prior to removal of the sample extraction device 252 optimized for VOCs, and then with subsequent insertion of a second sample extraction device 252 with internal sorbent element(s) optimized for SVOCs. Then, a strong vacuum is exerted on the hybrid sampling system 400, the vacuum source is removed, and the assembly is heated to 40-80 C to complete the transfer of SVOCs to sample extraction device 252 using “Vacuum Assisted Sorbent Extraction (VASE)”. In some cases, additional water can be added to the sample vessel 432 just prior to inserting extraction device 252, as when heating to 40-80 deg C., the additional water can create low pressure steam that can help to keep heavier SVOC off of the internal vessel surfaces (e.g., inner sidewall 436) to improve recovery on the sorbent element(s) in sample extraction device 252. Therefore, not only is sampler 400 immune to the effects of water in the sample, but additional water can also actually improve recoveries and operational performance of the samplers; purified water is an easy addition, and these properties demonstrate the unique nature of this sampling approach.

Then, both the VOC and the SVOC devices can be analyzed using a GC with the ideally suited column, which may be necessary when trying to achieve the very highest dynamic range in compound volatility. In most cases, required monitoring levels for SVOCs are lower than VOCs, as many are more dangerous, and they are typically at lower concentrations in stack gas and in human breath than are VOCs, so required analytical volumes needed are typically greater for SVOCs in order to reach required detection limits. Embodiments similar to the approach detailed above in which two sample extraction devices are used are described in greater detail below, in connection with FIGS. 6 and 7.

A shaft portion of vacuum sleeve 402 can either be occupied by a sample extraction device 252 (as illustrated), or vacant from any sample extraction devices (e.g., in embodiments where hybrid sampling system 400 is sometimes operated without such devices inserted into the vacuum sleeve). In some embodiments, the shaft portion of vacuum sleeve 402 has an inner surface with dimensions sized to accommodate the body of sample extraction device 252. In some embodiments, the inner surface of the shaft of sleeve 402 has dimensions that are separated from the body of sample extraction device 252 by a small gap.

Side port 412 can be an inlet port to the sample vessel 432 (similar to a sample vial), through which samples of a gas phase matrix can be collected into the sample vessel in some embodiments. Additionally, side port 412 can be an outlet port for drawing a vacuum into the inner chamber of sample vessel 432 (e.g., cavity defined by an inner sidewall 436 of the vessel), or any other evacuation of the contents of the sample vessel in other embodiments. As an example, side port 412 can sometimes be utilized to draw a vacuum into the inner chamber of sample vessel 432, by evacuating any gases within the inner sidewall 436 of the vessel (e.g., prior to a sampling period of the system 400).

In some embodiments, side port 412 can include a microvalve that can be selectively opened to allow for gases to travel from any gas source connected or coupled to the side port (e.g., the environmental/ambient air surrounding system 300, or a gas source otherwise coupled to the side port). In some embodiments, side port 412 can be in a closed state by default (which blocks the travel of gases in/out of sample vessel 432), or in an open state (allowing gases to travel in/out of sample vessel 432). In embodiments where samples of environmental or ambient gas phase matrices surrounding hybrid sampling system 400 are collected into sample vessel 432, side port 412 can be used to collect the samples by setting it to the open state for a duration of a collection or sampling interval.

Side port 412 can also be used to receive injections of standard gas phase matrices (e.g., samples of gases and VOCs/SVOCs in known concentrations/quantities) in some embodiments. As an example, 1 cc of a gas standard with known concentrations of certain given recovery compounds (or, known proportions of recovery compounds to gases), can be injected into sample vessel 432 via side port 412. In some embodiments, samples collected by a sample extraction device (e.g., devices 252-256) subsequent to the gas standard injection can be analyzed for the presence of the given recovery compounds, in addition to their concentrations. Analysis of the sample collected subsequent to the gas standard injection can be used to confirm (or alternatively, call into question) the proper sampling, proper storage, and proper recovery of compounds in the sample (e.g., the pre-existing sample within sample vessel 432, prior to the gas standard injection via side port 412). Liquid phase surrogate compounds for SVOC recovery validation can be added to the sampling system 400 after sampling has been completed, by momentarily removing extraction device 252 prior to performing evacuation and diffusive extraction at elevated temperatures.

Side port 412 can be coupled to attachments, such as a flow restrictor, flow regulator, or flow controller that attaches to the side port in some embodiments. When coupled to a flow restrictor, side port 412 can be in the “open state” to allow gases to travel in/out of sample vessel 432, while flow restrictor can be independently adjusted to change a rate at which gases are allowed to travel into the sample vessel. In some embodiments, side port 412 can be coupled to a flow restrictor, which is in turn connected to a gas source to be sampled by sample vessel 432 (e.g., a refinery gas stream, a process gas stream, a stack gas emission source, work place air, fenceline monitoring, ambient air, indoor air, an exhaled breath line of a ventilator system, and other gas phase matrices). As an example, a flow restrictor can be used to specify a rate at which samples from a gas source are collected into sample vessel 432 over a sampling interval in some embodiments. In some examples, a rate of vacuum pulled through a valve portion 214 of a sample extraction device 252 and/or 254 can be set to a relatively slow flow rate, to reduce the channeling effects that are exacerbated at relatively fast vacuum flow rates.

A threading region 416 of vacuum sleeve 402 can represent a lower portion of the vacuum sleeve that allows for the vacuum sleeve to be screwed into and fastened to any compatible threads, such as a threaded connector 408 (shown in FIG. 4A) that secures onto an opening of sample vessel 432 in some embodiments. In some embodiments, threaded connector 408 can either be an integrated part of the sample vessel 432, or a removable connector. In some embodiments, threaded connector 408 can be an integrated part of vacuum sleeve 402 (e.g., obviating the need for any threading between two separate components). A cap 422 can cover threaded connector 408 and secure it in position, thereby helping to retain vacuum sleeve 402 in its upright position after it is threaded onto the threaded connector in some embodiments.

Sample vessel 432 can have a portion 442 corresponding to a neck portion or an upper portion of the sample vessel, in some embodiments. Portion 442 can be tapered relative to other dimensions of sample vessel 432 (as illustrated), or can have any suitable geometry relative to the remainder of the sample vessel. Portion 444 can correspond to a lower portion or bottom portion of the sample vessel in some embodiments. Sample vessel 432 can have an inner sidewall 436 and an outer sidewall 434. Inner sidewall 436 can form a cavity into which gas samples from a gas phase matrix outside of sample vessel 432 can be collected. Water 350 within inner sidewall 436 can represent condensed water that is collected during the sampling process. Water taken into sample vessel 432 during sampling will saturate to 100% relative humidity within the sample vessel, and any excess will be liquid (e.g., as represented by water 350). In certain embodiments, liquid water may react with certain VOCs, and in such embodiments a sample extraction device 252 may have to be added to device system 400 prior to sampling, so the water reactive compounds can have a chance to be adsorbed by the sorbent element(s) of device 252 with reduced exposure time to the condensed water. In embodiments where compounds are known to not react with water (e.g., most compounds at ambient temperatures), as long as the compounds have a binding affinity with the sorbent that is stronger than their binding affinity with the water, near complete recovery into the sorbent is still possible when a sample extraction device 252 is added after the sampling period, given a reasonable amount of time for the compounds to find the sorbent element(s) of device 252 (e.g., via passive diffusive motion).

FIG. 4B illustrates a cross-sectional side view of a hybrid sampling system 450 for sampling gas phase matrices and collecting VOCs and SVOCs from the samples according to some embodiments of the disclosure, specifically illustrating sample extraction device 252 vertically offset from a side port 412 of vacuum sleeve 404 according to some embodiments of the disclosure. System 450 of FIG. 4B shows another representation of hybrid sampling system 400 of FIG. 4A, showing a similar sample extraction device 252 inserted into vacuum sleeve 406, such that device 252 is vertically offset from inlet 414 by a distance D1. In some embodiments, such an arrangement can allow for gas phase matrix samples to enter the system 450 to be collected through side port 412, without any of the gases and/or compounds carried by the gases to come into contact with sample extraction device 252, or any of its sidewalls.

FIG. 5 illustrates an exemplary process 500 for operating the hybrid sampling system to sample gas phase matrices according to some embodiments of the disclosure. To simplify descriptions associated with the process steps of process 500, reference is made to labeled components of hybrid sampling system 400 of FIG. 4A. However, it should be understood that in some embodiments, process 500 can be used for operating the hybrid sampling systems 400 or 450 of FIG. 4A or 4B without departing from the scope of the disclosed embodiments.

Process 500 begins at step 501, where sampling system 400 (sometimes referred to as a “sampler,” for simplicity) is assembled, the sampler including a sample extraction device (e.g., device 252), a sample vessel (e.g., vessel 432), and a vacuum sleeve (e.g., sleeve 402) that are all cleaned in a laboratory, and then assembled (e.g., according to the arrangements illustrated by FIG. 4A or 4B), in some embodiments. In particular, at step 501, cap 422 can be used to hold vacuum sleeve 402 onto the sample vessel 432, and retaining cap 404 can be used to keep sample extraction device 252 in the vacuum sleeve 402 from the time it leaves the lab to the time it is returned, in some embodiments. As mentioned above in connection with FIG. 4A, in certain embodiments, sampling system 400 can be used to sample gas phase matrices without a sample extraction device 252 present (or, inserted into vacuum sleeve 402) on-site, or in the field where the system is operate. In such embodiments, a solid retaining cap, or cap attachment 160 of FIG. 1B can be placed over vacuum sleeve 402 to create a closed system that is vacuum tight, prior to collecting gas samples through side port 412 of the vacuum sleeve.

After step 501, process 500 can proceed to step 502, which can occur after the hybrid sampling system is assembled, and prior to the system being sent to a sampling or collection site (e.g., a location where gas phase matrices of interest will be sampled by the system). At step 502, a vacuum can be drawn through sample extraction device 252 into a vacuum source via side port 412, to form a vacuum within the sample vessel 432. In some embodiments, after sample vessel 432 has been evacuated, a weight of the sample vessel 432 can be measured and recorded, to provide a baseline reference weight by which the weight of sampled gases and/or compounds can be determined after a sampling period at a sampling or collection site. Recording the sampler weight before and after sampling can also provide indications about the amount of water that condensed in the sampler, as the condensed water will not be a part of the gas phase sample, thereby increasing the relative concentrations of compounds still remaining in the gas phase. Moreover, a volume of the sample vessel can be changed and/or selected based on the required detection limits for a particular application, in some embodiments. As an example, for stack gas analysis where concentrations are expected to be at high parts-per-billion (PPB) through parts-per-million (PPM) levels, sample vessel 432 can have a 250 cc volume, and a sample collected by sample extracting device 252 can then be provided into the GCMS using a 50:1 split upon injection.

As described above, step 502 can be performed prior to the hybrid sampling system being sent to a sampling or collection site. Once the system arrives at the sampling or collection site, step 504 can be performed in some embodiments. At step 504, air samples containing bulk gases and compounds such as VOCs, SVOCs, etc. can be collected into sample vessel 432 over a time period (e.g., a sampling interval). At step 504, side port 412 of the sampling system is connected to a gas stream, after which a sample is introduced either rapidly (e.g., by opening the valve associated with the side port), or slowly using a flow restrictor or controller (e.g., in embodiments where a time integrated sample collection is required or desired). In some embodiments, when collection of heavier SVOCs is desired, or in order to keep steam from condensing prior to reaching the inside of sample vessel 432 a line providing the gas stream to be sampled, as well as side port 412 can be heated during the sampling time period or interval.

After the sampling time period or interval described in connection with step 504, the hybrid sampling system 400/450 can be returned to the laboratory for processing. In some embodiments, system 400/450 can be weighed and compared to the original evacuated weight (e.g., the weight measured after step 502) to determine the amount of liquid water collected, which in turn confirms that total amount of gas sampled when collecting from a high water containing sources at elevated temperatures. In some embodiments, this gravimetric determination can be used to verify whether 500 cc, 1000 cc, or even higher volumes were collected into a 500 cc reservoir, simply because some water may condense during the sampling process and before closing inlet 412, allowing more gas to be introduced. Once the sampling stops (e.g., by closing valve 412), any additional condensation of water only results in a reduction of the pressure in the vessel, and a final pressure measurement can reveal this occurrence, although gravimetric determination by looking at the weight gain of the sampler would be the best determination of the total mass collected, in some embodiments. In some embodiments, the actual volume sampled at the temperatures of the gas at the sampling point can then be easily calculated from the determined weight gain.

In some embodiments, after making this gravimetric determination, process 500 can proceed to step 506, where a vacuum is drawn through the top valve (e.g., valve end 214) and gases that are not retained by the sorbent element(s) in the sample extraction device (e.g., bulk, fixed gases) are removed from sample vessel 432. In some embodiments, a vacuum can be slowly applied to the top of sample extraction device 252 (e.g., where an internal seal comprising a sealing plunger and a seal is located, near valve end 214) to pull gas phase chemicals collected within sample vessel 432 onto the sorbent element(s) of the sample extraction device, thereby eliminating most of the fixed gases that are not retained on the sorbent. Evacuating bulk gases carrying compounds of interest (e.g., VOCs or SVOCs in the gas phase matrix) in this manner can improve collection/recovery rates of the compounds of interest onto the sorbent element(s) of one or more sample extraction devices during subsequent process steps. As a vacuum is drawn through valve end 214, the gases carrying VOCs and some SVOCs can travel through an extraction end 212 of device 252, through an internal channel that connects a lower cavity 220 or 222 to the valve end 214. During evacuation through the lower cavity 220 or 222, VOCs as well as some lighter SVOCs can be collected and/or recovered onto sorbent element(s) located in the lower cavity 220 or 222 (e.g., sorbent 202 or 256a-c of FIG. 2A or 2B). In some embodiments, it can be advantageous to maintain system 400 at a relatively low temperature (e.g., cooled) during step 506, to prevent losses through the sorbent for lighter compounds, as the system is “open,” by virtue of mass (e.g., of the removed gases) being removed from the system.

Process 500 can proceed to step 508, which occurs after step 506 in some embodiments. In other words, steps 506 and 508 can be considered as a sequence of steps, each associated with a respective temperature level provided for the respective sampling techniques described by those steps. As mentioned above, system 400 can be maintained at a relatively low temperature in connection with step 506. At step 508, compounds from the sampled air, or another gas phase matrix collected at step 504 (e.g., through side port 412), are adsorbed at one or more sorbent elements located in the lower cavity of a sample extraction device 252 (e.g., by entering an extraction end 212 of the device, and coming into contact with one or more sorbent elements). In some embodiments, step 508 can simply describe the adsorption of compounds collected at step 504 by one or more sorbent elements (e.g., omitting the optional heating of system 400).

In some embodiments, the entire sampling system 400 can be heated to raise vapor pressure of SVOCs within sample vessel 432, thereby promoting movement of said compounds towards the one or more sorbent elements of a sample extraction device 252 (e.g., as a vacuum is drawn through valve end 214 of the sample extraction device). In some embodiments, an extraction end 212 of the sample extraction device 252 can be heated relatively more than the overall/entire sampling system 400, to prevent or create conditions that discourage the collection of moisture in sample extraction device 252, by keeping sample vessel 432 slightly cooler than sample extraction device 252. In this way, step 508 can describe maintaining conditions that raise vapor pressure of SVOCs within sample vessel 432 that may be stuck along inner sidewall 436 of sample vessel 432, and promoting movement of SVOCs towards the one or more sorbent elements contained in sample extraction device 252. These conditions can include, in some embodiments, heating system 400 such that collected compounds within sample vessel 432 (e.g., VOCs, SVOCs) can come into contact with the sorbent element(s) of a sample extraction device 252 while a vacuum is drawn through sample extraction device 252 to collect the VOCs and SVOCs present in the air samples.

Because the compounds adsorbed by the sorbent element(s) are meant to be analyzed by GCMS and GC-MS/MS, these analysis techniques have some limitations, such as the potential for interference from excess water vapor. Many samples of complex gas phase matrices have elevated levels of water vapor, from ambient air which can be as much as 3-4% water vapor, to stack gas streams that may be 50% water vapor and at elevated temperatures. Another example of a complex gas phase matrix with elevated levels of water vapor is exhaled breath, with water vapor in exhaled breath exceeding 100% relative humidity at 37 degrees Celsius, due to aerosols and/or water droplets included in the exhaled breath. Excess water in the sample can cause damage to the GC column, and also suppression of the response in the MS. Therefore, water must be substantially reduced prior to injection into a GCMS or GC-MS/MS. Sampling and analysis techniques have been developed that can reduce water concentrations as long as the water is at non-condensing concentrations. As an example, if the dew point of the sample within sample vessel 432 is at 25 degrees Celsius and a sample extraction device 252 is at 35 degrees Celsius, then water can be kept in the gas phase thereby allowing it to pass through the one or more sorbent elements of the sample extraction device, unretained. However, in certain embodiments, sorbent element(s) cannot be generally increased in temperature to meet elevated gas stream temperatures, because doing so can prevent chemicals of interest from being trapped on the sorbent.

Process 500 can proceed to step 510, where water is extracted out of the sample extraction device and its sorbent element(s), to dehydrate the sorbent element(s). In some embodiments, step 510 can simply describe the dehydration of the sorbent element(s) (e.g., omitting the cooling of system 400). In some embodiments, a bottom portion 444 of sampling system 400 can be cooled to promote water condensation within sample vessel 432, and dehydrate the one or more sorbent elements of sample extraction device 252. In some embodiments, the sampling system 400 is placed on a cold tray to cool the bottom portion 444 of the sample vessel 432 to transfer most of the water in the closed system back to the bottom of the sample vessel (e.g., away from sample extraction device 252 and its sorbent element(s)). In some embodiments, bottom portion 444 can be cooled for between 5 and 30 minutes to transfer water in the closed system back to the bottom of the sample vessel. Moreover, at the lower temperatures, any of the very light VOCs that were not firmly maintained or adsorbed on the sorbent element(s) of device 252 can again collect onto one or more of the stronger beds of the sorbent element(s).

After step 510, process 500 can proceed to step 512, where sample extraction device 252 is removed from the vacuum sleeve by which it was coupled to sample vessel 432 (e.g., by first removing retention cap 404). Sample extraction devices with dehydrated sorbent element(s) can be removed from the hybrid sampling system 400 and can be isolated in individual sleeves (e.g., isolation sleeves 302 of FIG. 3), in some embodiments. In some examples, sample extraction devices with dehydrated sorbent element(s) can be analyzed by thermal desorption processes, using either a splitless injection technique, or a split injection technique. In embodiments where stack gases are monitored or measured by the hybrid sampling system 400, a split technique can usually be employed. In such embodiments, using a split injection to thermally desorb the sample extraction device can allow for total VOCs through SVOCs to be analyzed in a single analysis. In other embodiments, where low part per trillion measurements are needed for indoor or ambient air measurements may be required, splitless injections can also be performed.

The sequence of events described above in connection with FIG. 5 allows for VOCs and SVOCs of interest to be recovered, while rejecting excess water prior to GCMS analysis, in some embodiments. It is well understood that strengths of sorbents such as those contained in sample extraction device 252 decrease, as water condenses on them. VOCs can be mostly delivered to a sample extraction device while the sample contained in the sample vessel is optionally cooled, or actively chilled (e.g., step 510), thereby lowering water vapor pressure within the sample vessel, while also improving trapping efficiency for a one or more sorbent elements within sample extraction device 252. Further, by maintaining the sample extraction device 252 just 10 degrees Celsius higher than the temperature of the sample (e.g., during step 508) will keep the sorbent stronger than if no liquid water were on the sorbent, and dry enough to maintain the sorbent bed at or near full strength, as heating the sorbent 10 degrees Celsius higher can reduce the strength of the sorbent bed.

In some embodiments, SVOCs can be collected diffusively once a vacuum has been created (e.g., after step 506). When sorbents are heated, they expand, and when they are cooled, they contract. During contraction, gaps in the sorbent or along the walls can occur, causing inconsistent carrier gas or air flow through the sorbent, and allowing compounds to penetrate further into the sorbent bed, due to a lack of exposure to the entire bed. During diffusive compound collection or trapping under vacuum (e.g., during a collection interval), chemicals are allowed to travel in random directions rather than being steered in the direction of reducing pressure gradient, such as when gas is convectively flowing through a sorbent bed, and through channels in that sorbent bed. During static, diffusive sampling, the random movement of molecules allows them to distribute onto the sorbent bed in a true “affinity distribution” profile, with the very heaviest compounds right at the beginning of the sorbent bed, where they can be recovered the fastest, and where they are unlikely to create any background or carryover during the next analysis.

During the SVOC transfer stage, it is not necessary to heat the sampler to the boiling point of desired compounds in some embodiments. Even at relatively low temperatures (50-100 deg C.), compounds with boiling points of 400-600 deg C. will have some vapor pressure. Even if the equilibrium at a relatively low temperature is 99% adsorbed on the container walls, and 1% in the gas phase, that 1% will collect onto the sample extraction device, forcing yet another 1% to go into the gas phase (that retains the 100:1 adsorbed to gas phase ratio), and then another, until most or all of the high boiling compounds are transferred to the sample extraction device. This is in stark contrast to what is necessary during classical sampling of a headspace within a vial or container, where only the compounds that are in the headspace after a given equilibration period will be included in the analysis. Also, during vacuum extraction, boiling points of compounds can be reduced, allowing recovery of compounds at lower temperatures.

Even when using hydrophobic sorbent element(s) in sample extraction devices 252 and 254, some water will partition into the sample extraction devices when heated to higher temperatures, where water molecules will tend to randomize within the sample vessel 432. Therefore, after the heated extraction (e.g., step 508), and while the system is still a closed system, the bottom portion 444 of the vacuum container can be chilled (e.g., step 510) to a temperature well below that of the sample extraction device 252 at upper portion 442, and under vacuum any condensed water in the sample extraction device 252 can rapidly transfer out of the sorbent element(s) and back to the bottom of the vacuum container (e.g., water 449). Ultimately, VOCs and SVOCs have been transferred to the sorbent, while almost all of the condensed water has been left in the container to be removed during a subsequent cleaning or vessel evacuation process. After the extraction described by FIG. 5, the sample extraction device can be desorbed and collected compounds on their sorbent element(s) can be analyzed by GC or GCMS.

The sample extraction devices 252 and 254 can be cleaned enough for reuse after thermal desorption into a GCMS, without any additional cleaning in some embodiments. Sample vessel 432 and vacuum sleeve 402 can be rinsed in purified or deionized water, and then baked out either in a lab oven or heated under a vacuum to remove any additional water or chemical background. Heating the sample vessel and vacuum sleeve under a vacuum can be done prior to insertion of a sample extraction device 252, such as when optionally replacing a cap attachment 160 of FIG. 1B with the sample extraction device. Alternatively, a cap attachment 160 can be left in place, so that extraction using a sample extraction device 252 can be performed in the lab, later. When used properly, the sample extraction devices 252-256, vacuum sleeves 402, and sample vessels 432 can be used many hundreds of times, making process 500 an extremely cost effect solution over time.

FIG. 6 illustrates an exemplary process 600 for operating the hybrid sampling system to sample gas phase matrices using dual sample extraction devices according to some embodiments of the disclosure. To simplify descriptions associated with the process steps of process 600, reference is made to labeled components of hybrid sampling system 400 of FIG. 4A. However, it should be understood that in some embodiments, process 600 can be used for operating the hybrid sampling systems 400 or 450 of FIG. 4A or 4B without departing from the scope of the disclosed embodiments.

Process 600 can relate to embodiments in which two sample extraction devices are used, where a first sample extraction device containing first sorbent element(s) is optimized for adsorption of VOCs, and a second sample extraction device containing second sorbent element(s) is optimized for adsorption of SVOCs. In some embodiments, the first sample extraction device containing sorbent(s) optimized for VOC adsorption can be used in the field during air sample collection by the sample vessel 432. Since the gas phase samples and the VOCs contained within them are exposed to the first sample extraction device for an appreciable period of time (e.g., during the sampling, and subsequent transport back to a lab setting), passive and/or diffusive motion of the VOCs can occur at close to atmospheric pressure, over a prolonged period of time (e.g., 1 or more days). As described in greater detail below, vacuum assisted extraction can be performed in connection with the second sample extraction device (and optionally, with the first sample extraction device as well), so that SVOCs can be fully transferred to the second sample extraction device under a vacuum.

Process 600 begins at step 601, where sampling system 400 (sometimes referred to as a “sampler,” for simplicity) is assembled, the sampler including a sample extraction device (e.g., device 252) optimized for collection of VOCs through light SVOCs, a sample vessel (e.g., vessel 432), and a vacuum sleeve (e.g., sleeve 402) that are all cleaned in a laboratory, and then assembled (e.g., according to the arrangements illustrated by FIG. 4A or 4B), in some embodiments. In particular, at step 601, cap 422 can be used to hold vacuum sleeve 402 onto the sample vessel 432, and retaining cap 404 can be used to keep a sample extraction device 252 optimized for VOCs in the vacuum sleeve 402 from the time it leaves the lab to the time it is returned, in some embodiments. In the embodiment described by FIG. 6 a first sample extraction device containing first sorbent element(s) optimized for VOC adsorption and/or recovery can be held by a vacuum sleeve on sample vessel 432, and utilized on-site (e.g., allowing VOCs collected in the gas phase matrix samples to passively diffuse onto the first sorbent element(s) optimized for VOC recovery). In certain embodiments, sampling system 400 can be used to sample gas phase matrices without a sample extraction device 252 present (or, inserted into vacuum sleeve 402) on-site, or in the field where the system is operated. For process 600, placing the extraction device in the sampler after it reaches the lab would require the sampler to sit in the laboratory for one or more days to allow the diffusive transfer of the contents of the VOCs and light SVOCs to the sample extraction device. In such embodiments, a solid retaining cap, or cap attachment 160 of FIG. 1B can be placed over vacuum sleeve 402 to create a closed system that is vacuum tight, prior to collecting gas samples through side port 412 of the vacuum sleeve.

Step 602 can occur after the hybrid sampling system is assembled at step 601, and prior to the system being sent to a sampling or collection site (e.g., a location where gas phase matrices of interest will be sampled by the system). At step 602, a vacuum can be drawn through side port 412, to form a vacuum within the sample vessel 432. In some embodiments, after sample vessel 432 has been evacuated, a weight of the sample vessel 432 can be measured and recorded, to provide a baseline reference weight by which the weight of sampled gasses and/or compounds can be determined after a sampling period at a sampling or collection site.

As described above, step 602 can be performed prior to the hybrid sampling system being sent to a sampling or collection site. Once the system arrives at the sampling or collection site, step 604 can be performed in some embodiments. At step 604, similar to step 504 of FIG. 5, gas phase samples containing bulk gases and compounds such as VOCs, SVOCs, etc. can be collected into sample vessel 432 over a time period (e.g., a sampling interval). At step 604, side port 412 of the sampling system is connected to a gas stream, after which a sample is introduced either rapidly (e.g., by opening the valve associated with the side port), or slowly using a flow restrictor or controller (e.g., in embodiments where a time integrated sample collection is required or desired). In some embodiments, when collection of heavier SVOCs is desired, or in order to keep steam from condensing prior to reaching the inside of sample vessel 432 a line providing the gas stream to be sampled, as well as side port 412 can be heated during the sampling time period or interval.

After the sampling time period or interval described in connection with step 604, the hybrid sampling system 400 can be returned to the laboratory for processing. In some embodiments, system 400 can be weighed and compared to the original evacuated weight (e.g., the weight measured after step 602) to determine the amount of liquid water collected, which in turn confirms that total amount of gas sampled when collecting from a high water containing sources at elevated temperatures.

In some embodiments, process 600 can proceed to step 606, which describes adsorbing the compounds collected in the air samples within sample vessel 432 by passive or diffusive transfer, at the one or more sorbent elements of the sample extraction device. In some embodiments, step 606 can take place over a collection interval spanning any suitable amount of time required to transport a sample vessel coupled to sample extraction device to a laboratory for analysis (e.g., 1 day, 2 days, etc.). In connection with the first sample extraction device with first sorbent element(s) optimized for VOCs, a vacuum does not need to be drawn through valve end 214, unlike step 506 of FIG. 5, due to the prolonged duration of passive diffusion of collected compounds onto the first sorbent element(s) being considered sufficient for sample recovery onto the first sample extraction device, in the context of FIG. 6.

Process 600 can proceed to step 608, which is similar to step 510 of FIG. 5. At step 608, water is extracted out of the sample extraction device and its sorbent element(s), to dehydrate the sorbent element(s). In some embodiments, step 608 can simply describe the dehydration of the sorbent element(s) (e.g., omitting the cooling of system 400). In some embodiments, the entire sampling system 400 can be cooled to promote water condensation within sample vessel 432, and dehydrate the one or more sorbent elements of sample extraction device 252.

Process 600 can proceed to step 610, after step 608 has been performed for the first sample extraction device. At step 610, the first sample extraction device is replaced with the second sample extraction device containing second sorbent element(s) optimized for collection/recovery of SVOCs. After replacing the first sample extraction device with the second sample extraction device, process 600 can proceed to step 612.

At step 612, which is similar to step 506 of FIG. 5, in the context of the second sample extraction device containing second sorbent element(s), a vacuum is drawn through the top valve (e.g., valve end 214) and gases are removed from sample vessel 432. In some embodiments, a vacuum can be slowly applied to the top of sample extraction device 254 (e.g., where an internal seal comprising a sealing plunger and a seal is located, near valve end 214) to pull gas phase chemicals collected within sample vessel 432 onto the sorbent element(s) of the sample extraction device, thereby eliminating most of the fixed gases that are not retained on the sorbent. In general, most gas phase compounds and/or chemicals of interest will have already been collected by the first extraction device, so step 612 mostly ensures that the system is under vacuum, to allow faster diffusion of the heavier compounds mostly contained on the surface of the vessel once evacuation stops, and the resulting closed system can be heated in the next step (e.g., step 614).

At step 614, compounds from the sampled air, or another gas phase matrix collected at step 604 are adsorbed at one or more sorbent elements located in the lower cavity of a sample extraction device 254 (e.g., by entering an extraction end 212 of the device, and coming into contact with one or more sorbent elements). In some embodiments, step 614 can simply describe the adsorption of compounds collected at step 504 by one or more sorbent elements (e.g., omitting the optional heating of system 400). In some embodiments, the entire sampling system 400 can be heated to raise vapor pressure of SVOCs within sample vessel 432, thereby promoting movement of said compounds towards the one or more sorbent elements of a sample extraction device 252. In some embodiments, an extraction end 212 of the sample extraction device 252 can be heated relatively more than the overall/entire sampling system 400, to particularly encourage or promote movement of SVOCs within sample vessel 432 towards the extraction end (and the one or more sorbent elements contained within the lower cavity of the sample extraction device).

Process 600 can continue to step 616, similar to step 510 of FIG. 5 in the context of the second sample extraction device with second sorbent element(s), where water is extracted out of the sample extraction device and its sorbent element(s), to dehydrate the sorbent element(s). In some embodiments, step 616 can simply describe the dehydration of the second sorbent element(s) (e.g., omitting the cooling of system 400). In some embodiments, the sampling system 400 is placed on a cold tray to cool the bottom portion 444 of the reservoir to transfer most of the water in the closed system back to the bottom of the vacuum reservoir (e.g., away from sample extraction device 252 and its sorbent element(s)).

After step 616, process 600 can proceed to step 618, where the second sample extraction device 254 is removed from the vacuum sleeve by which it was coupled to sample vessel 432 (e.g., by first removing retention cap 404). Both the first sample extraction device and the second sample extraction device discussed above in connection with FIG. 6 can detect and recover compounds corresponding to VOCs through light SVOCs, and relatively heavier SVOCs, respectively. Sample extraction devices (e.g., the first/second sample extraction devices) with dehydrated sorbent element(s) (e.g., the first/second sorbent element(s)) can be removed from the hybrid sampling system 400 and can be isolated in individual sleeves (e.g., isolation sleeves 302 of FIG. 3), in some embodiments. In some examples, sample extraction devices with dehydrated sorbent element(s) can be analyzed by thermal desorption processes, using either a splitless injection technique, or a split injection technique. In embodiments where stack gases are monitored or measured by the hybrid sampling system 400, a split technique can usually be employed. In such embodiments, using a split injection to thermally desorb the sample extraction device can allow for total VOCs through SVOCs to be analyzed in a single analysis. In other embodiments, where low part per trillion measurements are needed for indoor or ambient air measurements may be required, splitless injections can also be performed.

FIG. 7 illustrates an exemplary process for operating the hybrid sampling system to sample gas phase matrices using dual sample extraction devices and different compound recovery processes for different sample extraction devices according to some embodiments of the disclosure. To simplify descriptions associated with the process steps of process 700, reference is made to labeled components of hybrid sampling system 400 of FIG. 4A. However, it should be understood that in some embodiments, process 700 can be used for operating the hybrid sampling systems 400 or 450 of FIG. 4A or 4B without departing from the scope of the disclosed embodiments.

Process 700 can relate to embodiments in which two sample extraction devices are used, where a first sample extraction device containing first sorbent element(s) is optimized for adsorption of VOCs through light SVOCs, and a second sample extraction device containing second sorbent element(s) is optimized for adsorption of SVOCs. In some embodiments, neither the first sample extraction device containing sorbent(s) optimized for adsorbing VOCs through light SVOCs, nor the second extraction device containing sorbent(s) optimized for adsorbing SVOCs are used in the field, during sample collection by the sample vial 432. Instead, a sampler similar to sampling system 170 of FIG. 1B provided with cap attachment 160 that seals an opening of the vacuum sleeve without requiring a co-present sample extraction device. As described in greater detail below, vacuum assisted extraction can be performed in connection with both the first sample extraction device, and the second sample extraction device.

Process 700 begins at step 701, where sampling system 400 (sometimes referred to as a “sampler,” for simplicity) is assembled, the sampler including a sample vessel (e.g., vessel 432), and a vacuum sleeve (e.g., sleeve 402) that are all cleaned in a laboratory, and then assembled (e.g., according to the arrangements illustrated by FIG. 4A or 4B), in some embodiments. In particular, at step 701, cap 422 can be used to hold vacuum sleeve 402 onto the sample vessel 432, and a cap attachment 160 similar to the one shown in connection with sampling system 170 of FIG. 1B can be used to keep vacuum sleeve 402 sealed to the environment, from the time it leaves the lab to the time it is returned, in some embodiments.

Step 702 can occur after the sampling system is assembled at step 701, and prior to the system being sent to a sampling or collection site (e.g., a location where gas phase matrices of interest will be sampled by the system). At step 702, a vacuum can be drawn through side port 412, to form a vacuum within the sample vessel 432.

As described above, step 702 can be performed prior to the vacuum sampling system being sent to a sampling or collection site. Once the system arrives at the sampling or collection site, step 704 can be performed in some embodiments. At step 704, similar to step 504 of FIG. 5 and step 604 of FIG. 6, gas phase samples containing bulk gases and compounds such as VOCs, SVOCs, etc. can be collected into sample vessel 432 over a time period (e.g., a sampling interval).

After the sampling time period or interval described in connection with step 704, the vacuum sampling system 170 can be returned to the laboratory for processing. In some embodiments, process 700 can proceed to step 706, in which pressure in sample vessel 432 is adjusted by introducing and/or adding an inert gas to the sample vessel, via side port 412. In some embodiments, the inert gas can be Ultra High Purity Nitrogen, or UHP N₂. In some embodiments, an amount of Nitrogen is added to adjust pressure within sample vessel 432 to atmospheric pressure.

After adding the inert gas to adjust pressure within sample vessel 432 to atmospheric pressure, process 700 can proceed to step 708, which describes coupling a first sample extraction device with one or more sorbent elements optimized for adsorption of VOCs to the sample vessel via vacuum sleeve 402/406.

Process 700 can then proceed to step 710, where a vacuum can be drawn through the top valve (e.g., valve end 214) and a known volume of gases are removed from sample vessel 432, specifically through sample extraction device 254 of FIG. 2B. In some embodiments, this gas extraction through device 254 can be done by using a volumetric measuring device such as a metering pump, a mass flow controller set to a known sampling rate for a known period of time, or using a volumetric device with a pressure sensor to meter a known volume of gas through sample extraction device 254. This will provide an accurate measurement of all compounds in the gas phase in the vessel (VOCs and light SVOC), while a secondary device afterwards can measure the heavier compounds primarily remaining on the walls of the sample vessel, using a vacuum, diffusive sampling process.

In some embodiments, a vacuum can be slowly applied to the top of sample extraction device 254 (e.g., where an internal seal comprising a sealing plunger and a seal is located, near valve end 214) to pull gas phase chemicals collected within sample vessel 432 onto the sorbent element(s) of the sample extraction device, thereby eliminating most of the fixed gases that are not retained on the sorbent. In some embodiments, the entire sampling system 400 can be optionally heated to raise vapor pressure of SVOCs within sample vessel 432, thereby promoting movement of said compounds towards the one or more sorbent elements of a sample extraction device 254 (e.g., as a vacuum is drawn through valve end 214 of the sample extraction device).

Process 700 can then proceed to step 712, which is similar to step 706 described above. At step 712, pressure in sample vessel 432 is adjusted by introducing and/or adding an inert gas to the sample vessel, via side port 412. In some embodiments, the inert gas can be Nitrogen, or N₂. In some embodiments, an amount of Nitrogen is added to adjust pressure within sample vessel 432 to atmospheric pressure. In some embodiments, step 712 can be performed so that virtually no outside air is introduced into the sampler when exchanging sample extraction devices (e.g., as described below, in connection with step 714).

Process 700 can then proceed to step 714, which describes replacing a first sample extraction device with first sorbent element(s) with a second sample extraction device with second sorbent element(s). In other words, step 714 describes removing the first sample extraction device from the vacuum sampler (and placing it in an isolation sleeve of FIG. 2, for example), and inserting the second sample extraction device into the vacuum sampler.

After replacing the first sample extraction device with the second sample extraction device, process 700 can proceed to step 716. At step 716, which is similar to step 506 of FIG. 5, in the context of the second sample extraction device containing second sorbent element(s), a vacuum is drawn through the top valve (e.g., valve end 214) and gases are removed from sample vessel 432. In some embodiments, a vacuum can be slowly applied to the top of sample extraction device 254 (e.g., where an internal seal comprising a sealing plunger and a seal is located, near valve end 214) to pull gas phase chemicals collected within sample vessel 432 onto the sorbent element(s) of the sample extraction device, thereby eliminating most of the fixed gases that are not retained on the sorbent. Similar to process 600, the lighter compounds collected during this process will have already been collected for quantitative measurement using the first extraction device (e.g., at step 710), so the retention of these compounds remaining in the gas phase will not be necessary on the sorbents in second sample extraction device 252, as this second device will be used to analyze the compounds that will become volatile and will transfer during the following vacuum assisted sorbent extraction (e.g., at step 718).

At step 718, compounds from the sampled air, or another gas phase matrix collected at step 704 are adsorbed at one or more sorbent elements located in the lower cavity of a sample extraction device 252 (e.g., by entering an extraction end 212 of the device, and coming into contact with one or more sorbent elements).

Process 700 can proceed to step 720, which is similar to step 510 of FIG. 5. At step 720, water is extracted out of the second sample extraction device and its sorbent element(s), to dehydrate the sorbent element(s). In some embodiments, step 720 can simply describe the dehydration of the sorbent element(s) (e.g., omitting the cooling of system 400). In some embodiments, the entire sampling system 400 can be cooled to promote water condensation within sample vessel 432, and dehydrate the one or more sorbent elements of sample extraction device 254.

After step 720, process 700 can proceed to step 722, where the second sample extraction device 252 is removed from the vacuum sleeve by which it was coupled to sample vessel 432 (e.g., by first removing retention cap 404). Both the first sample extraction device and the second sample extraction device discussed above in connection with FIG. 7 can detect and recover compounds corresponding to VOCs through light VOCs, and relatively heavier SVOCs, respectively. Sample extraction devices (e.g., the first/second sample extraction devices) with dehydrated sorbent element(s) (e.g., the first/second sorbent element(s)) can be removed from the hybrid sampling system 400 and can be isolated in individual sleeves (e.g., isolation sleeves 202 of FIG. 2), in some embodiments. In some examples, sample extraction devices with dehydrated sorbent element(s) can be analyzed by thermal desorption processes, using either a splitless injection technique, or a split injection technique. In embodiments where stack gases are monitored or measured by the hybrid sampling system 400, a split technique can usually be employed. In other embodiments, where low part per trillion measurements are needed for indoor or ambient air measurements may be required, splitless injections can also be performed.

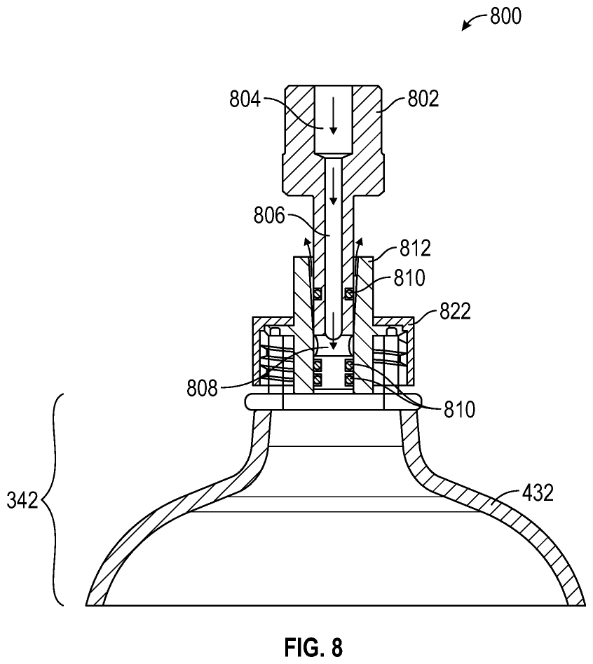

FIG. 8 illustrates an exemplary breath sampling system 800 with a breath inlet in the “Flow Divert” position according to some embodiments of the disclosure. System 800 illustrates an exemplary breath sampling system including a breath sampler inlet 802 held in place over a sample vessel 432 by a tapered attachment adapter 812 according to some embodiments of the disclosure. Breath sampler inlet 802 can include an inlet opening 804, coupled to an inlet shaft 806, which terminates at a high flow port 808, that directs air traveling down through inlet shaft 806 to the sides. Breath sampler inlet 802 can also include external seals 810, that can prevent air provided at inlet opening 804 from entering sample vessel 432 when the breath sampler inlet 802 is positioned as illustrated in FIG. 8 (e.g., with the bottom two external seals 810 creating a seal between the tapered attachment adapter 812 and the breath sampler inlet 802).

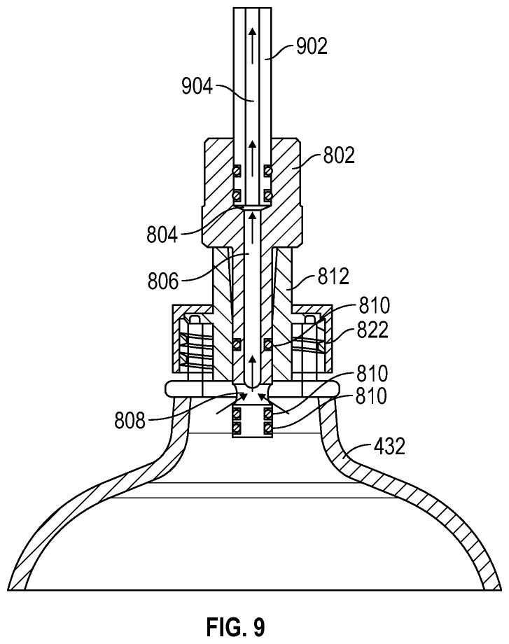

Breath sampler inlet 802 can sometimes be referred to as being in a “flow divert” position when configured or positioned as shown in FIG. 8, due to the seal between the breath sampler inlet 802 and the tapered attachment adapter 812 created by the bottom two external seals 810 sealing off the contents of vessel 432 from the environment. In some embodiments, if a patient were to exhale into breath sampler inlet 802 while in the up “Flow Divert” position (as shown in FIG. 9), air traveling down inlet opening 804 and inlet shaft 806 would be diverted to the sides, by high flow port 808, and be diverted away from sample vessel 432 (e.g., by traveling upwards, along the tapers of tapered attachment adapter 812). The taper in adapter 812 therefore allows the upper O-ring 810 to “not seal” when in the Up or Divert positions, but the two lower 810 O-rings prevents any breath from reaching vessel 832 while in the “Flow Divert” position (illustrated in FIG. 8).

FIG. 9 illustrates the exemplary breath sampling system 800 of FIG. 8 with the breath sampler inlet 802 in the “Flow Open” position to allow pre-evacuation or breath sample collection according to some embodiments of the disclosure. FIG. 9 also illustrates a vacuum source 902 with a vacuum inlet 904 coupled to breath sampler inlet 802 by external seals of the vacuum source 902 that create a seal between vacuum source 902 and an inner surface of inlet opening 804. In some embodiments, vacuum source 902 can be referred to as an “evacuation tool.” In addition to having vacuum source 902 seated within inlet opening 804, breath sampler inlet 802 can be positioned in the “flow open” position, by being pushed down toward the sample vessel 432. Downwards pressure on breath sampler inlet 802 can cause the inlet to move down such that the two bottom seals slide past the end of adapter 812, allowing the high flow port 808 in the inlet 802 to become pneumatically connected to vessel 432, while at the same time the upper O-ring seal 810 progresses down past the taper in adapter 812 so a seal is made to avoid pneumatic connection with the outside of inlet 802 (as illustrated in FIG. 9). As illustrated by FIG. 9, to initially evacuate sample vessel 432, a vacuum source 902 can be seated within inlet opening 804, and pressure can be applied downwards on breath sampler inlet 802 to initially evacuate sample vessel 432 using the vacuum source 902. A side port vacuum gauge can be connected to the vacuum source 902, and can verify that the sample vessel 432 is under vacuum, in some embodiments. In the position illustrated in FIG. 9, high flow port 808 creates a path between vacuum source 902 and vacuum inlet 904 to the inside of sample vessel 432, which can be evacuated using the vacuum source.

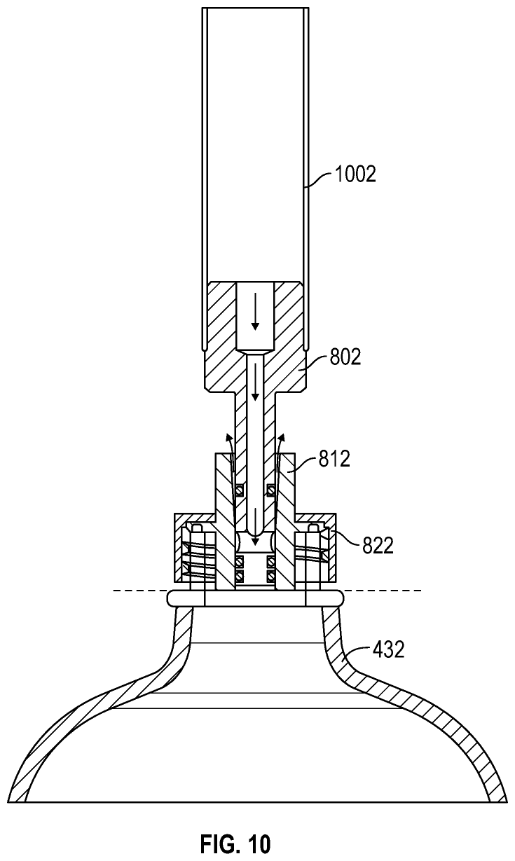

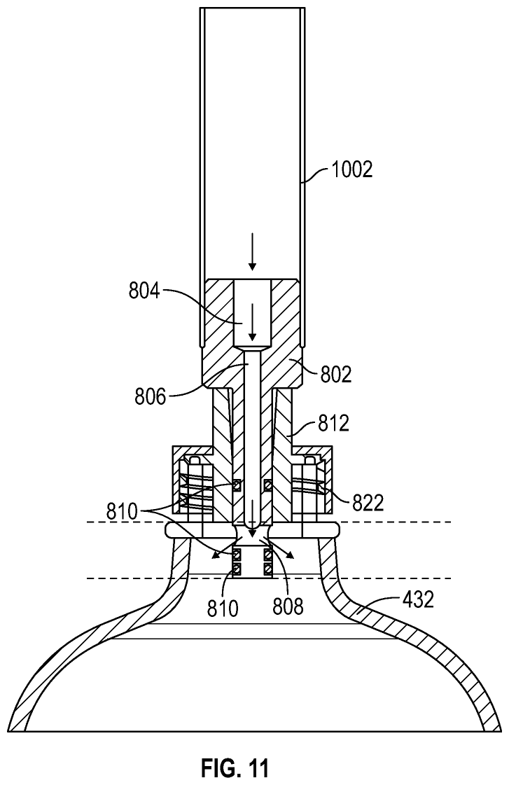

FIG. 10 illustrates the exemplary breath sampling system 800 with the breath sampler inlet 802 in the “Flow Divert” position coupled with a disposable mouth piece 1002 attached in the up “Ready to Sample” position according to some embodiments of the disclosure. Sampling system 800 of FIG. 10 can be similar to system 800 shown in FIG. 8. FIG. 10 differs from FIG. 8 in showing a disposable mouth piece 1002 coupled to breath sampler inlet 802. In some embodiments, disposable mouth piece 1002 is an optional component that is coupled to breath sampler inlet 802 for directing a patient's exhaled breath into the breath sampler. The position of breath sampler inlet 802 shown in FIG. 10, where the seal between the breath sampler inlet 802 and the tapered attachment adapter 812 created by the bottom two external seals 810 prevents any breath exhaled into the sampler from being able to enter sample vessel 432 (e.g., because high flow port 808 is located above the dashed line indicating the opening of sample vessel 432). The position of breath sampler inlet 802 shown in FIG. 10 can, in some embodiments, also be used for removal of the first part of the exhaled breath, sometimes referred to as “non-alveolar saturated breath.” In some embodiments, roughly 2-5 seconds of the patient's breath can be directed into breath sampler inlet 802 in the “Flow Divert” position shown by FIG. 10, before pushing down on breath sampler (e.g., as shown in FIG. 11) to collect “deep alveolar air,” or a remaining fraction of a patient's exhalation.

FIG. 11 illustrates the exemplary breath sampling system 800 with the breath sampler inlet 802 in the “Flow Open” position coupled with the disposable mouthpiece in the down “Collecting Breath” position according to some embodiments of the disclosure. Sampling system 800 of FIG. 11 can be similar to system 800 shown in FIG. 9. FIG. 11 differs from FIG. 9 in showing a disposable mouth piece 1002 coupled to breath sampler inlet 802, rather than a vacuum source 902. In some embodiments, disposable mouth piece 1002 is an optional component that is coupled to breath sampler inlet 802 for directing a patient's exhaled breath into the breath sampler. In some embodiments, breath sampling inlet 802 is a plastic, disposable part that acts both as the inlet and the disposable mouth piece, that is only used to collect breath from one patient before being disposed of.

In FIG. 9, breath sampler inlet 802 is illustrated in the “Flow Open” position to evacuate sample vessel 432. By contrast, in FIG. 11, breath sampler inlet 802 is illustrated in the “Flow Open” position to collect breath samples corresponding to a fraction of a patient's exhalation corresponding to “deep alveolar air” that is exhaled roughly 2-5 seconds after a patient has exhaled through the up, “Flow Divert” position of the breath sampler inlet. Downwards pressure on breath sampler inlet 802 can cause the inlet to move down such that the two bottom seals 810 slide past the end of adapter 812, allowing the high flow port 808 in the breath sampler inlet 802 to become pneumatically connected to vessel 432, while at the same time the upper O-ring seal 810 progresses down past the taper in adapter 812 so a seal is made to avoid pneumatic connection with the outside of inlet 802 (as illustrated). When the two lower seals 810 of breath sampler inlet 802 sliding past the end of adapter 812, such that an almost instant pneumatic connection between breath sampler inlet 802 to the sample vessel 432 forms. Connecting breath sampler inlet 802 to the sample vessel 432 in this way, can divert the alveolar saturated breath into sample vessel 432, carrying along with it water droplets and aerosols. that carry heavier organic compounds. The organic compounds can provide important information about metabolic processes in the body. The heavier compounds and aerosols/droplets are often missed by other sampling techniques, but can be successfully captured in the present sampler. When disposable mouth piece 1002 is coupled to breath sampler inlet 802 and positioned as illustrated in FIG. 11, the disposable mouth piece can be said to be in the down “Collecting Breath” position. Additionally, downwards pressure on breath sampler inlet 802 can further cause breath sampler inlet 802 to travel downwards, such that high flow port 808 is within an opening of sample vessel 432 (e.g., between the two dashed lines shown by FIG. 11). As illustrated by FIG. 11, to fill sample vessel 432 with a patient's exhaled breath, a disposable mouth piece 1002 can be seated within inlet opening around breath sampler inlet 802, and pressure can be applied downwards on breath sampler inlet 802 to fill vessel 432 using the patient's exhalation received via the mouth piece.

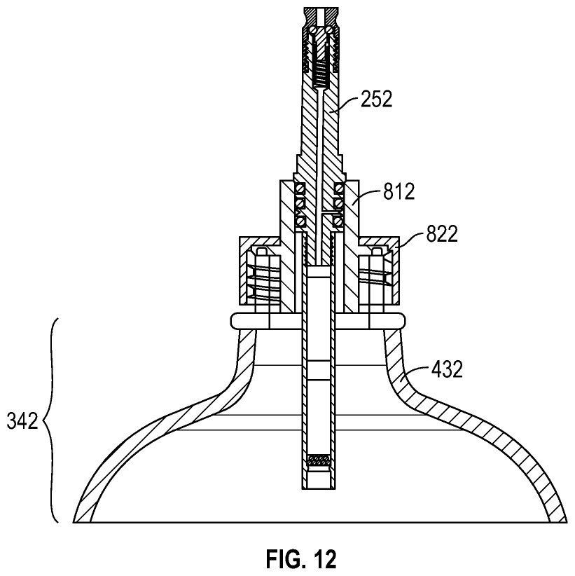

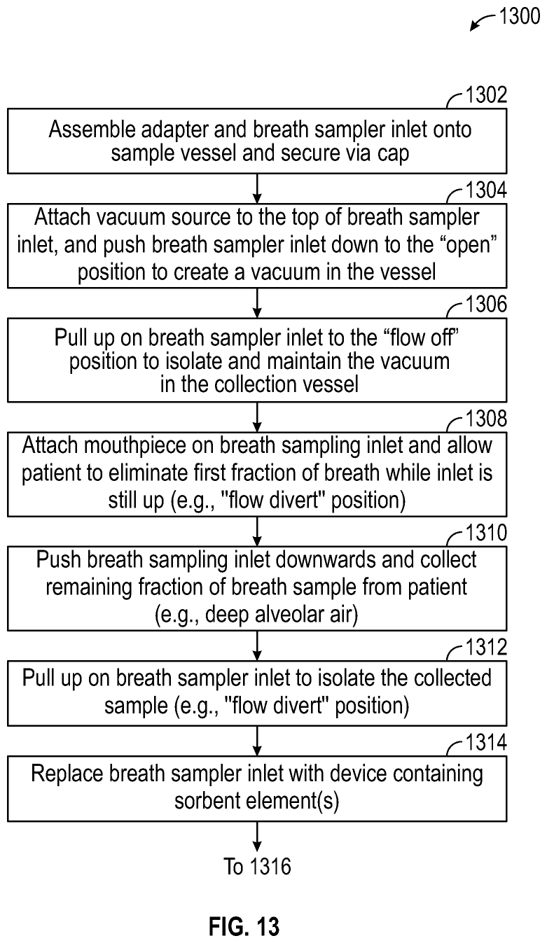

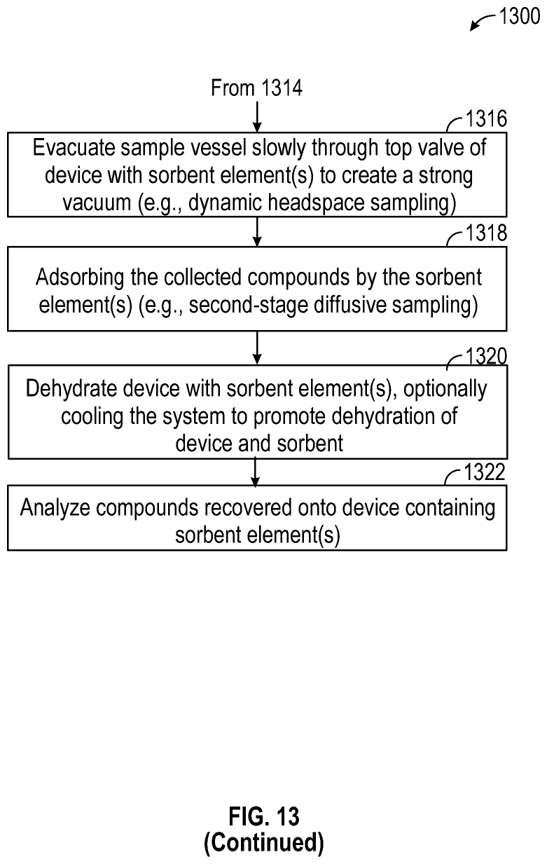

FIG. 12 illustrates the exemplary breath sampling system 800 with the breath inlet and the disposable mouthpiece replaced with a sample extraction device 252 according to some embodiments of the disclosure. An arrangement illustrated by FIG. 12 can be used to recover compounds from a patient's exhalation collected into sample vessel 432 onto a sample extraction device. As discussed in greater detail below in connection with FIG. 13, a vacuum can be drawn through a valve in a top portion of the sample extraction device, to evacuate contents of sample vessel 432 through the sample extraction device. Optionally, the system 800 of FIG. 12 can be heated in an oven to warm to between 40 and 80 degrees Celsius to improve collection of SVOCs from the patient's breath sample within sample vessel 432. Device 252 is shown inserted into the tapered attachment adapter 812 used to hold breath sampler inlet 802 in FIGS. 8-11.

FIG. 13 illustrates an exemplary process for operating a breath sampling system to collect breath samples and recover compounds from the samples according to some embodiments of the disclosure. To simplify descriptions associated with the process steps of process 1300 of FIG. 13, reference is made to labeled components of breath sampling system 800 of FIGS. 8-12.

Process 1300 begins at step 1302, which describes assembling an adapter 812 and breath sampler inlet 802 onto sample vessel 432, and securing the parts via a cap 822. Sampling system 800 (sometimes referred to as a “sampler,” for simplicity) is assembled, using sample vessel 432, and adapter 812, and breath sampler inlet 802 that are all cleaned in a laboratory, and then assembled (e.g., according to the arrangements illustrated by FIGS. 8-12), in some embodiments. An example of an assembled sample vessel with breath sampler inlet 802 can be seen in at least the configuration of breath sampling system 800, as illustrated by FIG. 8.

After step 1302, process 1300 can proceed to step 1304, which describes attaching a vacuum source 902 to the top of breath sampler inlet 802, and pushing breath sampler inlet down to the “flow open” position to create a vacuum in the vessel. Sampling system 800 can be configured to have a vacuum source 902 be seated within an inner surface of inlet opening 804. A side port vacuum gauge can be connected to the vacuum source 902, and can verify that the sample vessel 432 is under vacuum, in some embodiments. In the position illustrated in FIG. 9, high flow port 808 can create a path between vacuum source 902 and vacuum inlet 904 to the inside of sample vessel 432, which can be evacuated using the vacuum source when the breath sampler inlet 802 is in the “flow open position”,” as illustrated by FIG. 9.

After step 1304, process 1300 can proceed to step 1306, which describes pulling up on breath sampler inlet 802 to the “divert” position to isolate and maintain the vacuum in the collection vessel. In some embodiments, the position and/or configuration of breath sampler inlet 802 described by step 1304 is illustrated by FIG. 10. In some embodiments, the position and/or configuration of breath sampler inlet 802 described by step 1304 is illustrated by FIG. 8.

After step 1306, the process 1300 can proceed to step 1308, which describes attaching a mouthpiece (e.g., disposable mouthpiece 1002 of FIG. 10) on breath sampling inlet 802, and allowing patient to eliminate first fraction of breath while inlet is still up (e.g., “flow divert” position). As described above in connection with FIGS. 8 and 10, breath sampler inlet 802 can be positioned as shown in FIG. 10, where the seal between the breath sampler inlet 802 and the tapered attachment adapter 812 created by the bottom two external seals 810 prevents any breath exhaled into the sampler from being able to enter sample vessel 432 (e.g., because high flow port 808 is located above the dashed line indicating the opening of sample vessel 432). The position of breath sampler inlet 802 shown in FIG. 10 can, in some embodiments, also be used for removal of the first part of the exhaled breath, sometimes referred to as “non-alveolar saturated breath.” In some embodiments, roughly 2-5 seconds of the patient's breath can be directed into breath sampler inlet 802 in the “Flow Divert” position shown by FIG. 10, before pushing down on breath sampler to collect “deep alveolar air,” or a remaining fraction of a patient's exhalation (e.g., as shown in the next step at step 1310).

After step 1308, process 1300 can proceed to step 1310, which describes pushing breath sampling inlet 802 downwards and collect remaining fraction of breath sample from patient (e.g., deep alveolar air, as mentioned above in connection with the description of FIG. 11). In particular, step 1310 can refer to transitioning the breath sampling system from a first state (e.g., similar to the illustration of FIG. 10) in which none of a patient's initial breath sample/fraction are collected into a sample vessel, to a second state of the illustration of the FIG. 11 in which a patient's final breath sample/fraction of an exhalation is collected in to the sample vessel.

After step 1310, process 1300 can proceed to step 1312, which describes pulling breath sampler inlet up to isolate the collected sample (e.g., “flow divert” position). In some embodiments step 1312 can be illustrated by the breath sampling system of FIG. 10. After step 1312, process 1300 can proceed to step 1314, which describes replacing breath sampler inlet with device containing sorbent element(s), as illustrated by FIG. 12.

After step 1314, process 1300 can proceed to step 1316, which describes using dynamic headspace sampling to evacuate sample vessel 432 through a top valve of sample extraction device 252. In this manner, a known volume of sample can be drawn through the sorbent bed(s) during sample extraction, and a vacuum can be formed within sample vessel 432. In particular compounds from the deep alveolar air from a patient's exhalation, or another gas phase matrix collected at step 1310 are adsorbed at one or more sorbent elements located in the lower cavity of a sample extraction device 252 (e.g., by entering an extraction end 212 of the device, and coming into contact with one or more sorbent elements).

After step 1316, process 1300 can proceed to step 1318, which describes using second-stage diffusive sampling techniques to adsorb compounds from the deep alveolar air from a patient's exhalation, or another gas phase matrix collected at step 1310.

After step 1318, process 1300 can proceed to step 1320, which describes dehydrating the sample extraction device with at least one sorbent element, optionally cooling the system to promote dehydration of device and sorbent. In particular, at step 1318, a bottom portion 444 of sample vessel 432 can be cooled to promote water condensation within sample vessel 432, and dehydrate the one or more sorbent elements of sample extraction device 252.

After step 1318, process 1300 can proceed to step 1320, which describes analyzing compounds recovered onto device containing at least one sorbent element. In some examples, sample extraction devices with dehydrated sorbent element(s) can be analyzed by thermal desorption processes, using either a splitless injection technique, or a split injection technique.

In some embodiments, processes 500, 600, and 700, can be used in connection with the breath sampling system operations described by process 1300, except that the sample extraction device 252 may not be placed in the container during sample collection (e.g., during step 1310). In connection to steps described in connection with process 500, a single extraction device can be placed in the sample vessel 432, a vacuum can be slowly pulled on the container containing a sample of a patient's exhalation (slowly to avoid channeling), and once under vacuum, the sample vessel 432 can be heated to allow a second stage vacuum diffusive transfer of heavy compounds to the extraction device without likewise transferring the heavy, non-volatile compounds to the extraction device 252 (proteins, carbohydrates, lipids, bacteria), and without accumulating water on the sample extraction device 252.

In connection to steps described in connection with process 600, a sample extraction device 252 can be placed in the breath sampling system 800 (e.g., seated within adapter 812, as illustrated by FIG. 12) to diffusively collect the VOCs and light SVOCs over a 1 day or more period, followed by removal of the sample extraction device 252 to place a second extraction device in adapter 812, followed by evacuation and heating to recover the heavier SVOCs. The VOC filled extraction device can be desorbed into a GCMS optimized for VOC analysis, while the second extraction device desorbed into a GCMS optimized for SVOC analysis.

Finally, in connection to steps described in connection with process 700 an active sampling extraction device 254 can be used by withdrawing a known volume, then restoring atmospheric pressure using UHP N2 through side port 412, followed by exchanging the first extraction device for a second extraction device optimized for SVOC analysis, where a vacuum is created in breath sampling system 800 through the top of the second extraction device, followed by heating to perform a diffusive, vacuum recovery of the heavier SVOCs in the breath sample, while leaving the non-volatile compounds in vessel 432 where they will not create artifacts when the extraction devices are desorbed into a GCMS

Therefore, in some embodiments, techniques disclosed herein can be used in the analysis of chemicals in gas phase matrices sampled under conditions ranging from ambient temperatures to 300 degrees Celsius, and moisture concentrations from 0 to 50 percent. In some embodiments, a method comprises at a vessel with an attached extraction device containing sorbent, performing the following steps. In some embodiments, the method comprises creating a pre-sampling vacuum in the vessel using a vacuum inlet coupled to the vessel. In some embodiments, the method further comprises collecting, by the vessel, a gas phase sample into the vessel. In some embodiments, the method further comprises removing, through an opening of the attached extraction device, a volume of gas from the vessel while collecting one or more first compounds of the gas phase sample with the sorbent via dynamic headspace sampling technique. In some embodiments, the method further comprises disconnecting a vacuum source from the opening of the attached extraction device to create a closed system. In some embodiments, the method further comprises performing a second stage diffusive extraction under a partial to strong vacuum, to collect one or more second compounds. In some embodiments, the method further comprises heating the vessel during the second stage diffusive extraction to improve recovery of one or more low volatility compounds. In some embodiments, the method further comprises after detaching the vacuum inlet from the vessel, collecting one or more second compounds of the gas phase sample with the sorbent via a diffusive sampling technique.

In some embodiments, the opening of the attached extraction device comprises an upper opening on a first end of the attached extraction device, wherein the attached extraction device has a lower opening on a second end of the attached extraction device that is opposite the first end, wherein the first end of the attached extraction device is located outside of the vessel, and wherein the second end of the attached extraction device is located within the vessel. In some embodiments, the vessel further comprises a vacuum sleeve having an inner cavity that forms a seal with the attached extraction device such that the second end of the attached extraction device is under vacuum after the partial to strong vacuum is created in the vessel. In some embodiments, the second end of the attached extraction device is positioned above an inlet of the vacuum sleeve that allows gases to travel in and out of the vessel along a path that is separated from the extraction device.

In some embodiments, creating the pre-sampling vacuum comprises coupling the vacuum inlet to a side port valve of a vacuum sleeve that couples the attached extraction device to the vessel, and evacuating, using the vacuum inlet coupled to the side port valve, the vessel through the side port. In some embodiments, the method further comprises detaching the attached extraction device, and replacing the detached extraction device with a second extraction device. In some embodiments, the sorbent of the attached extraction device comprises at least a first sorbent element optimized to collect volatile compounds (VOCs), and wherein the second extraction device comprises at least a second sorbent element optimized to collect semi-volatile compounds (SVOCs). In some embodiments, the method further comprises adding, by a side port valve of a vacuum sleeve that couples the attached extraction device to the vessel, inert gas to the vessel, prior to replacing the detached extraction device with the another extraction device. In some embodiments, the method further comprises dehydrating the sorbent after collecting the one or more first compounds of the gas phase sample via the dynamic headspace sampling technique or after the second stage diffusive extraction to collect one or more second compounds, and before removing the attached extraction device from the vessel.

In some embodiments, the vessel has an upper portion to which the attached extraction device is coupled, and a lower portion opposite the upper portion, and wherein dehydrating the sorbent comprises cooling at least the lower portion of the vessel. In some embodiments, collecting the gas phase sample comprises opening a side port valve of a vacuum sleeve that couples the extraction device to the vessel to allow gases to enter the vessel through an inlet of the vacuum sleeve, without the gases contacting the attached extraction device while entering the vessel. In some embodiments, the method further comprises collecting the gas phase sample through the side port via a minimal path length inlet that is heated to keep moisture from over-condensing prior to reaching the vessel. In some embodiments, collecting the gas phase samples comprises receiving, at the vessel, samples of a gas phase matrix from a valve coupled to a flow restrictor when time integrated sampling is required, or when the production of gas to be sampled occurs at a low rate. In some embodiments, collecting the gas phase samples comprises receiving, at the vessel, samples of a gas phase matrix heated between 0 and 300 degrees Celsius without affecting the recovery of VOCs and SVOCs in the gas phase samples. In some embodiments, collecting the gas phase samples comprises receiving, at the vessel, samples of a gas phase matrix with a water concentration between 0 and 50 percent. In some embodiments, the extraction device is coupled to the vessel using a vacuum sleeve, and wherein the method further comprises securing, by a retention cap configured to fit around the extraction device, a coupling between the extraction device and the vacuum sleeve, such that the extraction device is not removable from the vacuum sleeve when secured by the retention cap.

In some embodiments, a method can comprise, at a vessel with an adapter having an opening, seating a breath sampler inlet within the opening, and creating a pre-sampling vacuum in the vessel using a vacuum inlet coupled to the breath sampler inlet, by pushing down the breath sampler inlet into the opening, to pneumatically couple an interior of the vessel to the vacuum inlet. In some embodiments, the method can further comprise pulling up on the breath sampler inlet, to maintain the pre-sampling vacuum in the vessel. In some embodiments, the method can further comprise receiving, at a mouthpiece coupled to the breath sampler inlet, a first fraction of a breath sample corresponding to an exhalation, and eliminating the first fraction of the breath sample, while the breath sampler inlet is pulled up. In some embodiments, the method can further comprise pushing down on the breath sampler inlet to pneumatically couple the interior of the vessel to the mouthpiece, and receiving, at the mouthpiece, a second fraction of the breath sample corresponding to the exhalation. In some embodiments, the method can further comprise collecting, by the vessel, the second fraction of the breath sample, while the breath sampler inlet is pushed down. In some embodiments, the method can further comprise pulling up on the breath sampler inlet to isolate the collected second fraction of the breath sample, removing the breath sampler inlet from the opening of the adapter, and inserting a sample extraction device into the opening of the adapter.

In some embodiments, collecting the second fraction of the breath sample comprises receiving the fraction of a breath sample from a patient's exhalation through the breath sampler inlet with minimal loss of water droplets or aerosols in the breath that contain important, diagnostically relevant SVOCs. In some embodiments, the breath sampler inlet has a divert position corresponding to when the breath sampler inlet is pulled up, wherein the first fraction of the breath sample corresponds to non-alveolar air, wherein the breath sampler inlet has a sample collection position corresponding to when the breath sampler inlet is pushed down, and wherein the second fraction of the breath sample corresponds to deep alveolar air. In some embodiments, a path between the mouthpiece and the vessel is formed when the breath sampler inlet is pushed down, and wherein the path allows for zero-loss collection of VOCs and SVOCs in breath.

Although examples have been fully described with reference to the accompanying drawings, it is to be noted that various changes and modifications will become apparent to those skilled in the art. Such changes and modifications are to be understood as being included within the scope of examples of this disclosure as defined by the appended claims.

1. A method, comprising:
at a vessel with an attached extraction device containing sorbent:
creating a pre-sampling vacuum in the vessel using a vacuum inlet coupled to the vessel;
collecting, by the vessel, a gas phase sample into the vessel;
removing, through an opening of the attached extraction device, a volume of gas from the vessel while collecting one or more first compounds of the gas phase sample with the sorbent via dynamic headspace sampling technique;
disconnecting a vacuum source from the opening of the attached extraction device to create a closed system;
performing a second stage diffusive extraction under a partial to strong vacuum, to collect one or more second compounds;
heating the vessel during the second stage diffusive extraction to improve recovery of one or more low volatility compounds; and
after detaching the vacuum inlet from the vessel, collecting one or more second compounds of the gas phase sample with the sorbent via a diffusive sampling technique.

2. The method of claim 1, wherein the opening of the attached extraction device comprises an upper opening on a first end of the attached extraction device, wherein the attached extraction device has a lower opening on a second end of the attached extraction device that is opposite the first end, wherein the first end of the attached extraction device is located outside of the vessel, and wherein the second end of the attached extraction device is located within the vessel.

3. The method of claim 2, wherein the vessel further comprises a vacuum sleeve having an inner cavity that forms a seal with the attached extraction device such that the second end of the attached extraction device is under vacuum after the partial to strong vacuum is created in the vessel.

4. The method of claim 3, wherein the second end of the attached extraction device is positioned above an inlet of the vacuum sleeve that allows gases to travel in and out of the vessel along a path that is separated from the extraction device.

5. The method of claim 1, wherein creating the pre-sampling vacuum comprises:
coupling the vacuum inlet to a side port valve of a vacuum sleeve that couples the attached extraction device to the vessel; and
evacuating, using the vacuum inlet coupled to the side port valve, the vessel through the side port.

6. The method of claim 1, further comprising:
detaching the attached extraction device; and
replacing the detached extraction device with a second extraction device.

7. The method of claim 6, wherein the sorbent of the attached extraction device comprises at least a first sorbent element optimized to collect volatile compounds (VOCs), and wherein the second extraction device comprises at least a second sorbent element optimized to collect semi-volatile compounds (SVOCs).

8. The method of claim 6, further comprising:
adding, by a side port valve of a vacuum sleeve that couples the attached extraction device to the vessel, inert gas to the vessel, prior to replacing the detached extraction device with the second extraction device.

9. The method of claim 1, further comprising:
dehydrating the sorbent after collecting the one or more first compounds of the gas phase sample via the dynamic headspace sampling technique or after the second stage diffusive extraction to collect one or more second compounds, and before removing the attached extraction device from the vessel.

10. The method of claim 9, wherein the vessel has an upper portion to which the attached extraction device is coupled, and a lower portion opposite the upper portion, and wherein dehydrating the sorbent comprises:
cooling at least the lower portion of the vessel.

11. The method of claim 1, wherein collecting the gas phase sample comprises:
opening a side port valve of a vacuum sleeve that couples the extraction device to the vessel to allow gases to enter the vessel through an inlet of the vacuum sleeve, without the gases contacting the attached extraction device while entering the vessel.

12. The method of claim 11, further comprising:
collecting the gas phase sample through the side port via a minimal path length inlet that is heated to keep moisture from over-condensing prior to reaching the vessel.

13. The method of claim 1, wherein collecting the gas phase samples comprises:
receiving, at the vessel, samples of a gas phase matrix from a valve coupled to a flow restrictor when time integrated sampling is required, or when production of gas to be sampled occurs at a low rate.

14. The method of claim 1, wherein collecting the gas phase samples comprises:
receiving, at the vessel, samples of a gas phase matrix heated between 0 and 300 degrees Celsius without affecting the recovery of VOCs and SVOCs in the gas phase samples.

15. The method of claim 1, wherein collecting the gas phase samples comprises:
receiving, at the vessel, samples of a gas phase matrix with a water concentration between 0 and 50 percent.

16. The method of claim 1, wherein the extraction device is coupled to the vessel using a vacuum sleeve, and wherein the method further comprises:
securing, by a retention cap configured to fit around the extraction device, a coupling between the extraction device and the vacuum sleeve, such that the extraction device is not removable from the vacuum sleeve when secured by the retention cap.

17. A method, comprising:
at a vessel with an adapter having an opening:
seating a breath sampler inlet within the opening;
creating a pre-sampling vacuum in the vessel using a vacuum inlet coupled to the breath sampler inlet, by pushing down the breath sampler inlet into the opening, to pneumatically couple an interior of the vessel to the vacuum inlet;
pulling up on the breath sampler inlet, to maintain the pre-sampling vacuum in the vessel;
receiving, at a mouthpiece coupled to the breath sampler inlet, a first fraction of a breath sample corresponding to an exhalation;
eliminating the first fraction of the breath sample, while the breath sampler inlet is pulled up;
pushing down on the breath sampler inlet to pneumatically couple the interior of the vessel to the mouthpiece;
receiving, at the mouthpiece, a second fraction of the breath sample corresponding to the exhalation;
collecting, by the vessel, the second fraction of the breath sample, while the breath sampler inlet is pushed down;
pulling up on the breath sampler inlet to isolate the collected second fraction of the breath sample;
removing the breath sampler inlet from the opening of the adapter; and
inserting a sample extraction device into the opening of the adapter.

18. The method of claim 17, wherein collecting the second fraction of the breath sample comprises:
receiving the fraction of a breath sample from a patient's exhalation through the breath sampler inlet with minimal loss of water droplets or aerosols in the breath that contain important, diagnostically relevant SVOCs.

19. The method of claim 17, wherein the breath sampler inlet has a divert position corresponding to when the breath sampler inlet is pulled up, wherein the first fraction of the breath sample corresponds to non-alveolar air, wherein the breath sampler inlet has a sample collection position corresponding to when the breath sampler inlet is pushed down, and wherein the second fraction of the breath sample corresponds to deep alveolar air.

20. The method of claim 17, wherein a path between the mouthpiece and the vessel is formed when the breath sampler inlet is pushed down, and wherein the path allows for zero-loss collection of VOCs and SVOCs in breath.
